(19) United States
(12) Patent Application Publication
(10) Pub. No.: US 2023/0099858 A1
(43) Pub. Date: Mar. 30, 2023

(54) PYRIDOPYRIMIDINE DERIVATIVES AS KRAS INHIBITORS

(71) Applicant: BETA PHARMA, INC., Wilmington, DE (US)

(72) Inventors: Don Zhang, Princeton, NJ (US); Jirong Peng, Mequon, WI (US); Michael John Costanzo, Bonney Lake, WA (US); Michael Alan Green, Easton, PA (US); Michael Nicholas Greco, Lansdale, PA (US)

(21) Appl. No.: 17/797,452

(22) PCT Filed: Feb. 19, 2021
(86) PCT No.: PCT/US2021/018703

§ 371 Date: Aug. 4, 2022

Related U.S. Application Data
(60) Provisional application No. 62/978,954, filed on Feb. 20, 2020.

(51) Int. Cl. C07D 487/04; C07D 519/00

(52) U.S. Cl. CPC C07D 487/04; C07D 519/00

(57) ABSTRACT

The present invention is directed to inhibitors of Kirsten Rat sacoma virus (KRAS), and more particularly to compounds of Formula (I), as well as compositions comprising Formula I and methods of using the compound of Formula I for the treatment or prevention of a disease, disorder, or medical condition mediated through KRAS, especially the KRAS mutant G12C.

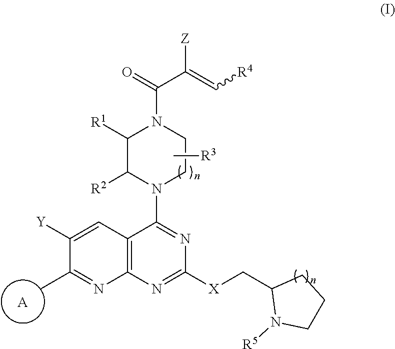

### CROSS-REFERENCE TO RELATED APPLICATIONS

This Application claims priority to U.S. Provisional Application Ser. No. 62/978,954 filed Feb. 20, 2020, which is herein incorporated by reference in its entirety.

### BACKGROUND OF THE INVENTION

### Field of the Invention

The present invention is directed to inhibitors of Kirsten Rat sarcoma virus (KRAS), and more particularly to pyridopyrimidine compounds, compositions and methods for the treatment or prevention of a disease, disorder, or medical condition mediated through KRAS, especially the KRAS mutant G12C. The diseases include various cancers.

### Brief Description of the Related Art

Ras is a superfamily of small guanosine triphosphate (GTP) binding proteins consisting of various isoforms. Ras genes can mutate to oncogenes that are associated with numerous cancers such as lung, pancreas, and colon. Ras is one of the most frequently mutated oncogenes. KRAS, (Kirsten Rat sarcoma virus) an isoform of Ras, is one of the most frequently mutated Ras genes, comprising approximately 86% of all mutations. KRAS functions as an on/off switch in cell signaling. KRAS is a proto-oncogene that operates between inactive (GDP-bound) and active (GTP-bound) states to control a variety of functions, including cell proliferation. However, KRAS mutation leads to uncontrolled cell proliferation and cancer. KRAS-4B is the major isoform in cancers of the colon (30-40%), lung (15-20%) and pancreas (90%). (Liu, P. 2019, Acta Pharmaceutica Sinica B). Consequently, inhibitors of KRAS-GTP binding represent potential therapeutic agents for the treatment of various cancers.

Past attempts to design KRAS inhibitors have been mostly unsuccessful, due in large part to the high affinity of KRAS for GTP. However, more recent approaches that target the KRAS G12C mutation have shown more promise. This mutation exists in roughly 50% of lung cancers and approximately 10-20% of all KRAS G12 mutations. The cysteine residue of the mutation is positioned within the active site such that the sulfhydryl functionality can form a covalent bond with a suitably functionalized bound ligand (Liu, P. 2019, Acta Pharmaceutica Sinica B). This approach has identified irreversible, covalent inhibitors of the KRAS G12C mutation that are undergoing clinical study. Given the prominent role that KRAS plays as a driver of many malignancies, a need for new KRAS inhibitors with improved selectivity, safety, and efficacy exists.

### SUMMARY OF THE INVENTION

In one aspect, the present invention is directed to a compound of Formula I:

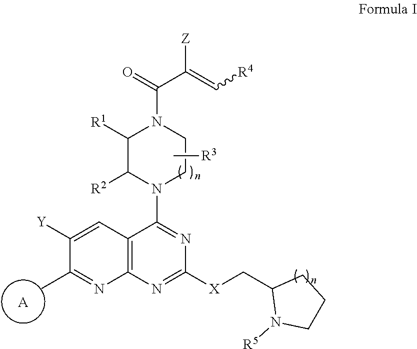

or a salt, solvate, or prodrug thereof, wherein
A is chosen from aryl or heteroaryl optionally substituted with one or more of hydrogen, halogen, hydroxyl, C₁₋₆alkyl, C₂₋₆alkenyl, C₂₋₆alkynyl, —(C₀₋₆alkyl)cycloalkyl, C₋₆haloalkyl, C₁₋₆ alkoxy, NO₂, cyano, CO₂H, PO(R⁶)₂, NH₂, NH(C₁₋₆alkyl) or N(C₁₋₆alkyl)₂;
X is chosen from O, NR⁷, S or CH₂;
Y is chosen from hydrogen, halogen or trifluoromethyl;
Z is chosen from hydrogen, halogen, trifluoromethyl or C₁₋₆alkyl;
R¹ is chosen from hydrogen, C₁₋₆alkyl, -(C₁₋₆alkyl)C₁₋₆alkoxy, —C₀₋₆alkyl(cycloalkyl), C₁₋₆haloalkyl, —(C₁₋₆alkyl)CN or —(C₁₋₆alkyl)P(O)(R⁶)₂;
R² is chosen from hydrogen, C₁₋₆alkyl, —(C₁₋₆alkyl)C₁₋₆alkoxy, —C₀₋₆alkyl(cycloalkyl), C₁₋₆haloalkyl, —(C₁₋₆alkyl)CN, —(C₁₋₆alkyl)P(O)(R⁶)₂ or taken together with R³ to represent 3-6 membered bridged ring;
R³ is chosen from hydrogen, C₁₋₆alkyl, —(C₁₋₆alkyl)C₁₋₆alkoxy, —C₀₋₆alkyl(cycloalkyl), C₁₋₆haloalkyl, —(C₁₋₆alkyl)CN, —(C₁₋₆alkyl)P(O)(R⁶)₂ or taken together with R² to represent 3-6 membered bridged alkyl ring;
n is 1-3;
R⁴ is chosen from hydrogen, halogen, C₁₋₃alkyl, C₁₋₃haloalkyl, —(C₁₋₃alkyl)CF₃ or —(C₁₋₃alkyl)C₁₋₆alkoxy;
R⁵ is chosen from H, C₁₋₆alkyl, C₃₋₆cycloalkyl or —(C₁₋₆alkyl)P(O)(R₆)₂;
R⁶ is chosen from H, C₁₋₆alkyl, C₃₋₆cycloalkyl, C₁₋₆alkoxy, C₃₋₆cycloalkoxy, hydroxyl, aryl or aryloxy;
R⁷ is chosen from H, C₁₋₆ alkyl, or C₃₋₆cycloalkyl.

In another aspect, the present invention is directed to a pharmaceutical composition comprising a compound of Formula I, or a salt, solvate, or prodrug thereof, together with a pharmaceutically acceptable carrier.

In another aspect, the present invention is directed to a method of treating a disease, disorder, or medical condition in a patient, comprising the step of providing to a patient in need thereof a therapeutic agent, wherein the therapeutic agent comprises the compound of Formula I or a salt, solvate, or prodrug thereof.

These and other aspects will become apparent upon reading the following detailed description of the invention.

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

All formulae disclosed herein include all salts of such Formulae.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁₋₈alkyl, C₁₋₄alkyl, and C₁₋₂alkyl. When C₀₋ᵤalkyl is used herein in conjunction with another group, for example, —C₀₋₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in —O—C₀₋₄alkyl(C₃₋₇cycloalkyl). Examples of alkyl include, but are not limited to, methyl, ethyl, n-propyl, isopropyl, n-butyl, 3-methylbutyl, t-butyl, n-pentyl, and sec-pentyl.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “Alkylthio” or a “thioalkyl” group is an alkyl group as defined above with the indicated number of carbon atoms covalently bound to the group it substitutes by a sulfur bridge (—S—). Similarly, “alkenyloxy”, “alkynyloxy”, and “cycloalkyloxy” refer to alkenyl, alkynyl, and cycloalkyl groups, in each instance covalently bound to the group it substitutes by an oxygen bridge (—O—).

“Halo” or “halogen” means fluoro, chloro, bromo, or iodo, and are defined herein to include all isotopes of same, including heavy isotopes and radioactive isotopes. Examples of useful halo isotopes include ¹⁸F, ⁷⁶Br, and ¹³¹I. Additional isotopes will be readily appreciated by one of skill in the art.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Peptide” means a molecule which is a chain of amino acids linked together via amide bonds (also called peptide bonds).

“Pharmaceutical compositions” means compositions comprising at least one active agent, such as a compound or salt of Formula II, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

### Chemical Description

Compounds of the Formulae disclosed herein may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, prodrugs, free base compound and salts) of the compounds of the invention may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

The term “solvate” refers to a chemical complex formed by the interaction of a solvent and a solute, such as the chemical compounds of the present invention.

The term “prodrug” refers to a biologically inactive compound which can be metabolized inside or outside the body to produce a drug.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

A “chelating group” or “chelator” is a ligand group which can form two or more separate coordinate bonds to a single central atom, which is usually a metal ion. Chelating groups as disclosed herein are organic groups which possess multiple N, O, or S heteroatoms, and have a structure which allows two or more of the heteroatoms to form bonds to the same metal ion.

“Salts” include derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts. In an embodiment, the compounds of the present invention are synthesized or isolated as trifluoroacetic acid (TFA) salts.

In one embodiment, the salt forms of the compounds of the present invention described above may include pharmaceutically acceptable salts. Examples of pharmaceutically acceptable salts include, but are not limited to, non-toxic mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

As indicated above, the compounds of the present invention relate to substituted pyridopyrimidine derivatives or salts, solvates, or prodrugs thereof, wherein the 4-amino group contains a functionality such as but-3-ene-2-one, as shown in Formula I:

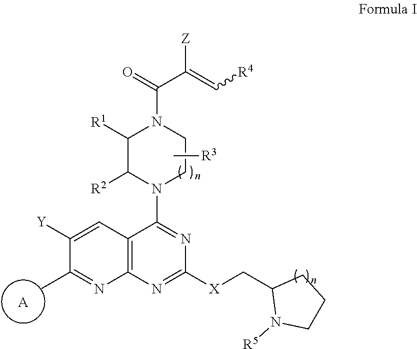

or a salt, solvate, or prodrug thereof, wherein
A is chosen from aryl or heteroaryl optionally substituted with one or more of hydrogen, halogen, hydroxyl, C₁₋₆alkyl, C₂₋₆alkenyl, C₂₋₆alkynyl, —(C₀₋₆alkyl)cycloalkyl, C₋₆haloalkyl, C₁₋₆ alkoxy, NO₂, cyano, CO₂H, PO(R⁶)₂, NH₂, NH(C₁₋₆alkyl) or N(C₁₋₆alkyl)₂;
X is chosen from O, NR⁷, S or CH₂;
Y is chosen from hydrogen, halogen or trifluoromethyl;
Z is chosen from hydrogen, halogen, trifluoromethyl or C₁₋₆alkyl;
R¹ is chosen from hydrogen, C₁₋₆alkyl, —(C₁₋₆alkyl)C₁₋₆alkoxy, —C₀₋₆alkyl(cycloalkyl), C₁₋₆haloalkyl, —(C₁₋₆alkyl)CN or —(C₁₋₆alkyl)P(O)(R⁶)₂;
R² is chosen from hydrogen, C₁₋₆alkyl, —(C₁₋₆alkyl)C₁₋₆alkoxy, —C₀₋₆alkyl(cycloalkyl), C₁₋₆haloalkyl, —(C₁₋₆alkyl)CN, —(C₁₋₆alkyl)P(O)(R⁶)₂ or taken together with R³ to represent 3-6 membered bridged ring;
R³ is chosen from hydrogen, C₁₋₆alkyl, —(C₁₋₆alkyl)C₁₋₆alkoxy, —C₀₋₆alkyl(cycloalkyl), C₁₋₆haloalkyl, —(C₁₋₆alkyl)CN, —(C₁₋₆alkyl)P(O)(R⁶)₂ or taken together with R² to represent 3-6 membered bridged alkyl ring;
n is 1-3;
R⁴ is chosen from hydrogen, halogen, C₁₋₃alkyl, C₁₋₃haloalkyl, —(C₁₋₃alkyl)CF₃ or —(C₁₋₃alkyl)C₁₋₆alkoxy;
R⁵ is chosen from H, C₁₋₆alkyl, C₃₋₆cycloalkyl or —(C₁₋₆alkyl)P(O)(R₆)₂;
R⁶ is chosen from H, C₁₋₆alkyl, C₃₋₆cycloalkyl, C₁₋₆alkoxy, C₃₋₆cycloalkoxy, hydroxyl, aryl or aryloxy;
R⁷ is chosen from H, C₁₋₆ alkyl, or C₃₋₆cycloalkyl.

In the preferred embodiments, the compounds of Formula I are represented by 1a-z and 2a-2n or a salt, solvate, or prodrug thereof:

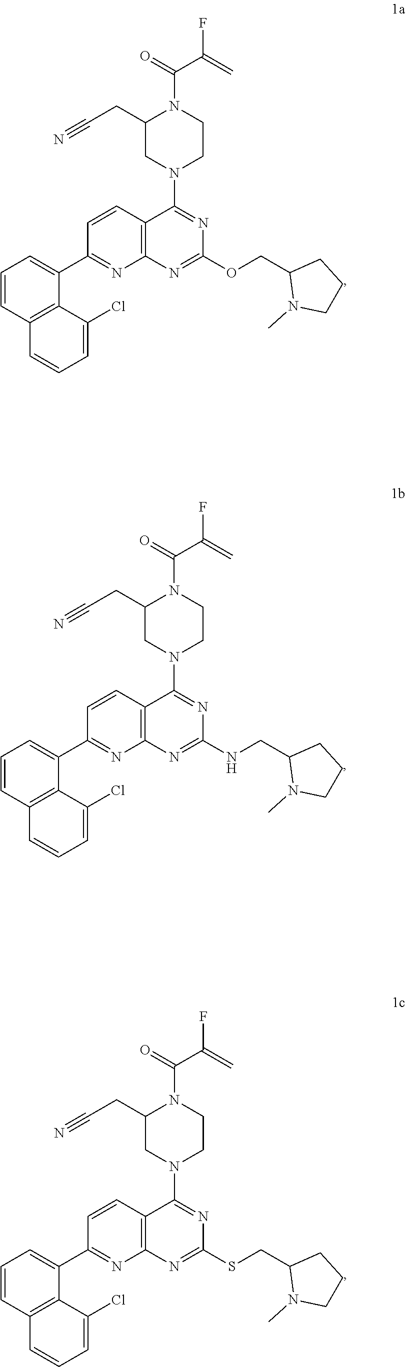 [SMILES: C=C(F)C(=O)N1CCN(c2nc(OCC3CCCN3C)nc3nc(-c4cccc5cccc(Cl)c45)ccc23)CC1CC#N ; C=C(F)C(=O)N1CCN(c2nc(NCC3CCCN3C)nc3nc(-c4cccc5cccc(Cl)c45)ccc23)CC1CC#N ; C=C(F)C(=O)N1CCN(c2nc(SCC3CCCN3C)nc3nc(-c4cccc5cccc(Cl)c45)ccc23)CC1CC#N]

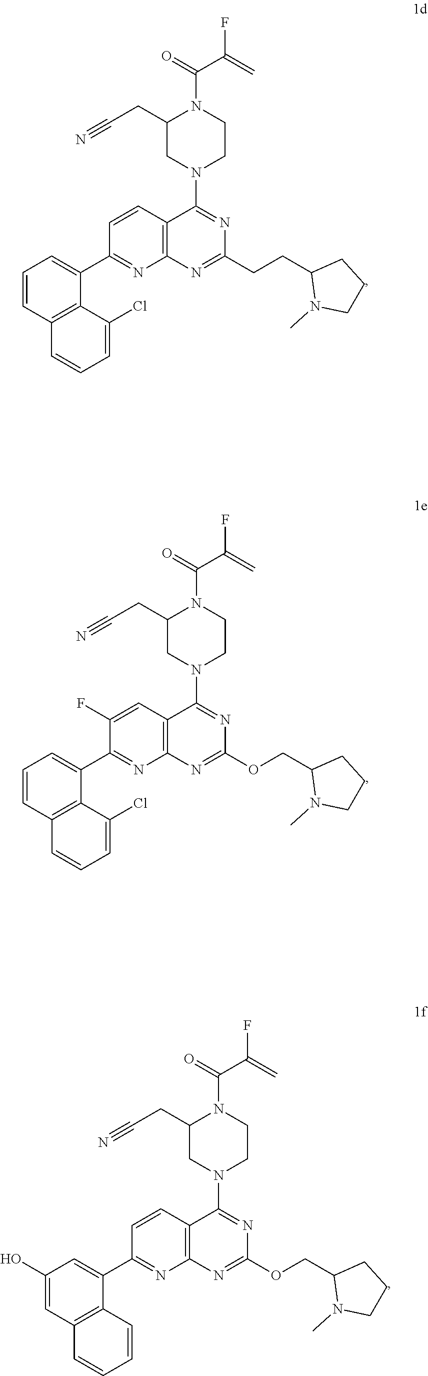 [SMILES: C=C(F)C(=O)N1CCN(c2nc(CCC3CCCN3C)nc3nc(-c4cccc5cccc(Cl)c45)ccc23)CC1CC#N ; C=C(F)C(=O)N1CCN(c2nc(OCC3CCCN3C)nc3nc(-c4cccc5cccc(Cl)c45)c(F)cc23)CC1CC#N ; C=C(F)C(=O)N1CCN(c2nc(OCC3CCCN3C)nc3nc(-c4cc(O)cc5ccccc45)ccc23)CC1CC#N]

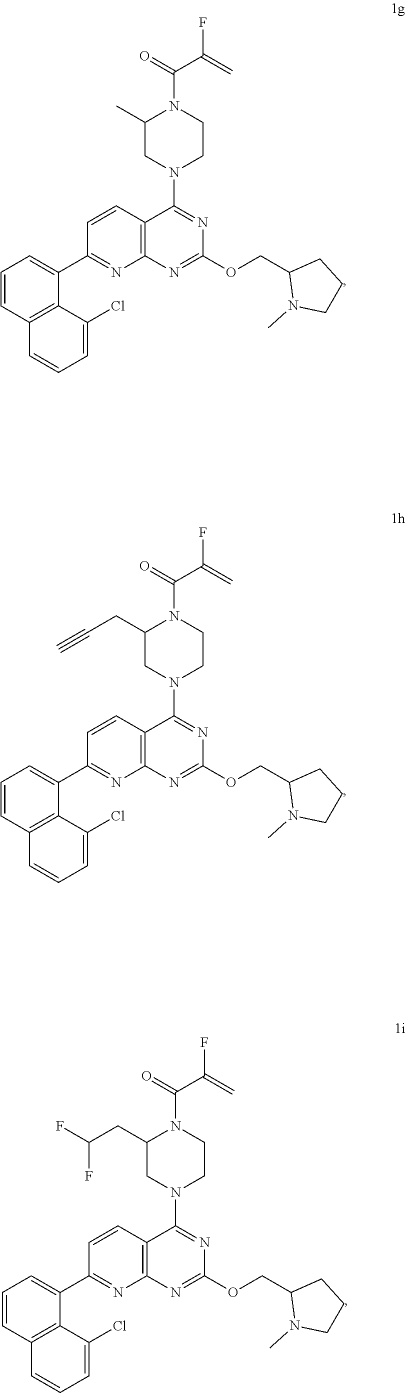 [SMILES: C=C(F)C(=O)N1CCN(c2nc(OCC3CCCN3C)nc3nc(-c4cccc5cccc(Cl)c45)ccc23)CC1C ; C#CCC1CN(c2nc(OCC3CCCN3C)nc3nc(-c4cccc5cccc(Cl)c45)ccc23)CCN1C(=O)C(=C)F ; C=C(F)C(=O)N1CCN(c2nc(OCC3CCCN3C)nc3nc(-c4cccc5cccc(Cl)c45)ccc23)CC1CC(F)F]

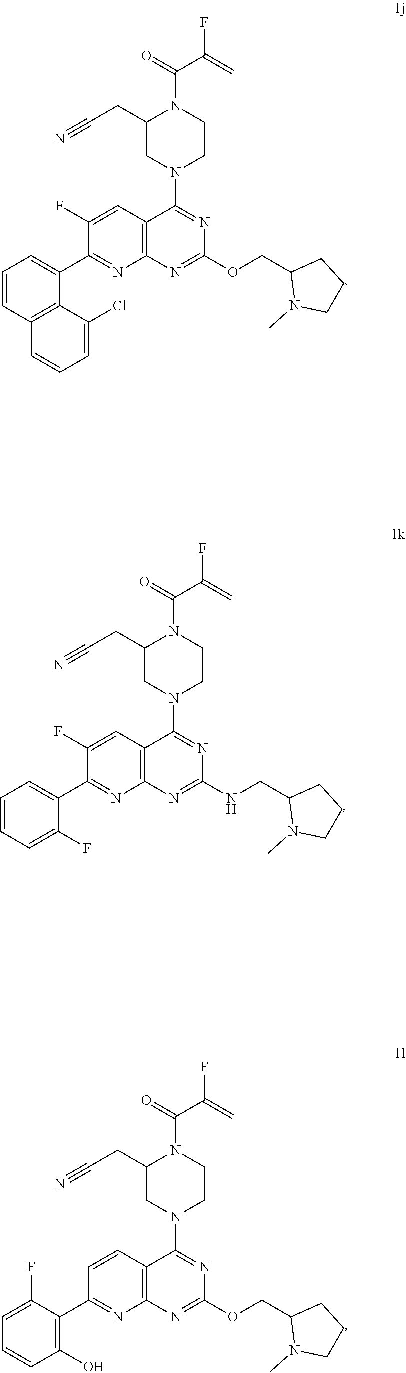 [SMILES: C=C(F)C(=O)N1CCN(c2nc(OCC3CCCN3C)nc3nc(-c4cccc5cccc(Cl)c45)c(F)cc23)CC1CC#N ; C=C(F)C(=O)N1CCN(c2nc(NCC3CCCN3C)nc3nc(-c4ccccc4F)c(F)cc23)CC1CC#N ; C=C(F)C(=O)N1CCN(c2nc(OCC3CCCN3C)nc3nc(-c4c(O)cccc4F)ccc23)CC1CC#N]

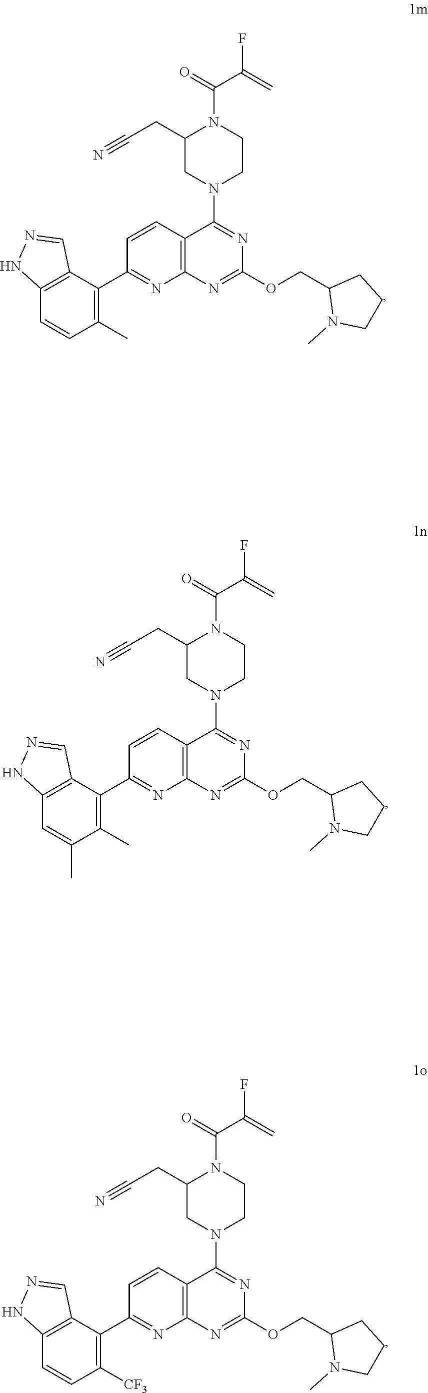 [SMILES: C=C(F)C(=O)N1CCN(c2nc(OCC3CCCN3C)nc3nc(-c4c(C)ccc5[nH]ncc45)ccc23)CC1CC#N ; C=C(F)C(=O)N1CCN(c2nc(OCC3CCCN3C)nc3nc(-c4c(C)c(C)cc5[nH]ncc45)ccc23)CC1CC#N ; C=C(F)C(=O)N1CCN(c2nc(OCC3CCCN3C)nc3nc(-c4c(C(F)(F)F)ccc5[nH]ncc45)ccc23)CC1CC#N]

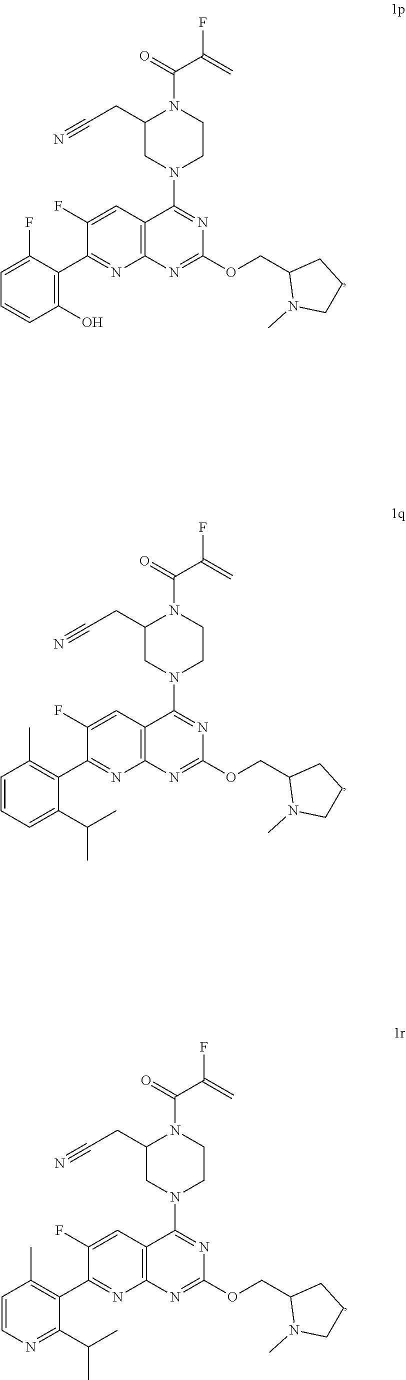 [SMILES: C=C(F)C(=O)N1CCN(c2nc(OCC3CCCN3C)nc3nc(-c4c(O)cccc4F)c(F)cc23)CC1CC#N ; C=C(F)C(=O)N1CCN(c2nc(OCC3CCCN3C)nc3nc(-c4c(C)cccc4C(C)C)c(F)cc23)CC1CC#N ; C=C(F)C(=O)N1CCN(c2nc(OCC3CCCN3C)nc3nc(-c4c(C)ccnc4C(C)C)c(F)cc23)CC1CC#N]

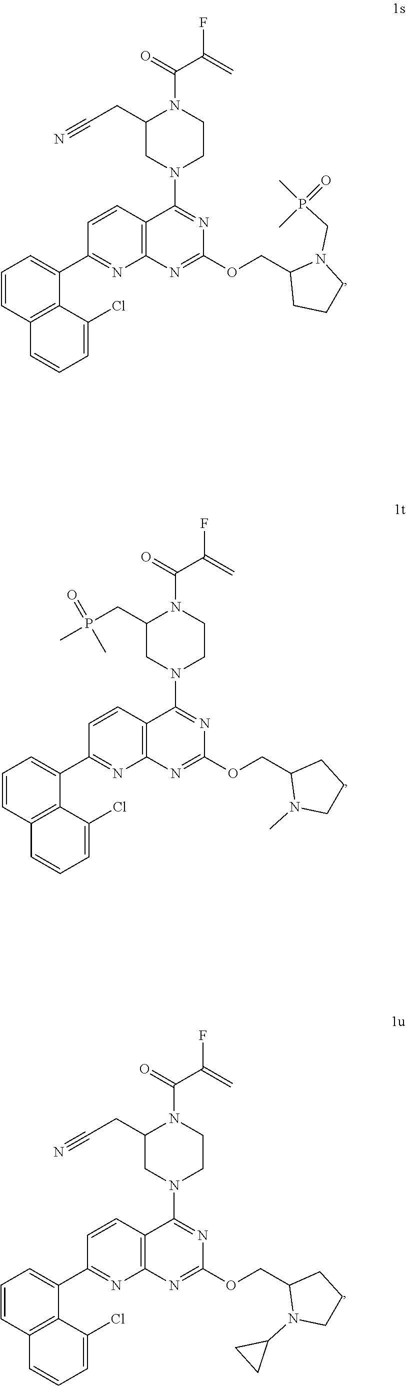 [SMILES: C=C(F)C(=O)N1CCN(c2nc(OCC3CCCN3CP(C)(C)=O)nc3nc(-c4cccc5cccc(Cl)c45)ccc23)CC1CC#N ; C=C(F)C(=O)N1CCN(c2nc(OCC3CCCN3C)nc3nc(-c4cccc5cccc(Cl)c45)ccc23)CC1CP(C)(C)=O ; C=C(F)C(=O)N1CCN(c2nc(OCC3CCCN3C3CC3)nc3nc(-c4cccc5cccc(Cl)c45)ccc23)CC1CC#N]

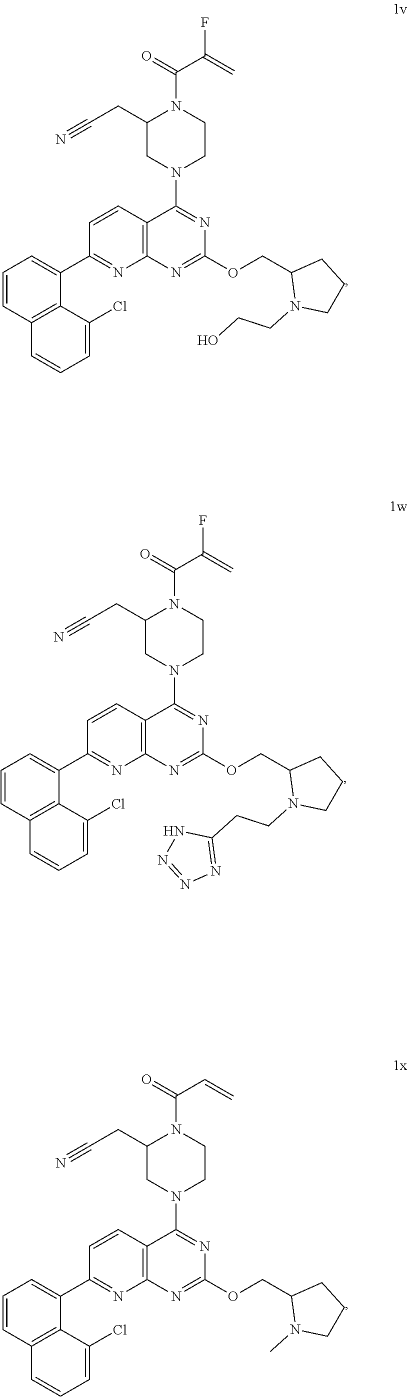 [SMILES: C=C(F)C(=O)N1CCN(c2nc(OCC3CCCN3CCO)nc3nc(-c4cccc5cccc(Cl)c45)ccc23)CC1CC#N ; C=C(F)C(=O)N1CCN(c2nc(OCC3CCCN3CCc3nnn[nH]3)nc3nc(-c4cccc5cccc(Cl)c45)ccc23)CC1CC#N ; C=CC(=O)N1CCN(c2nc(OCC3CCCN3C)nc3nc(-c4cccc5cccc(Cl)c45)ccc23)CC1CC#N]

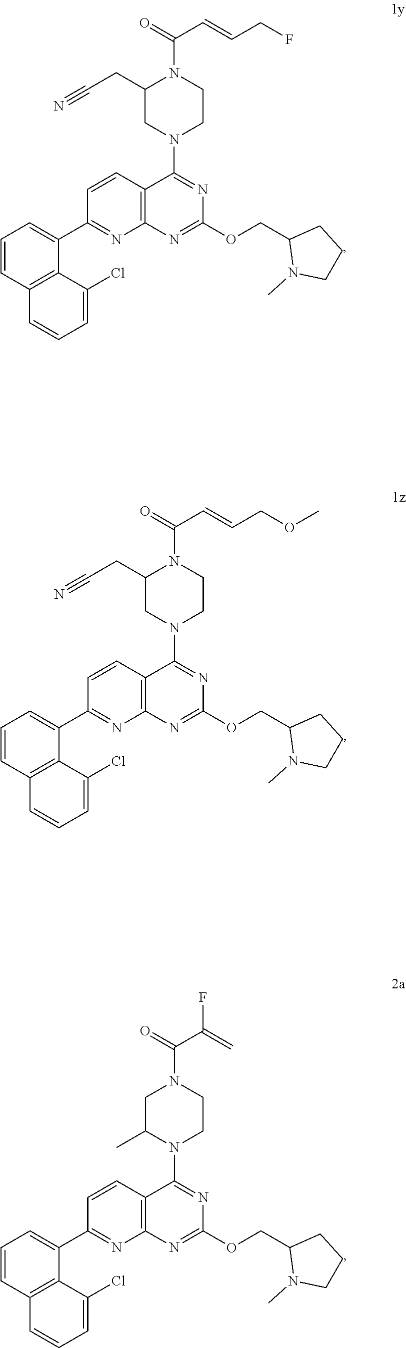 [SMILES: CN1CCCC1COc1nc(N2CCN(C(=O)/C=C/CF)C(CC#N)C2)c2ccc(-c3cccc4cccc(Cl)c34)nc2n1 ; COC/C=C/C(=O)N1CCN(c2nc(OCC3CCCN3C)nc3nc(-c4cccc5cccc(Cl)c45)ccc23)CC1CC#N ; C=C(F)C(=O)N1CCN(c2nc(OCC3CCCN3C)nc3nc(-c4cccc5cccc(Cl)c45)ccc23)C(C)C1]

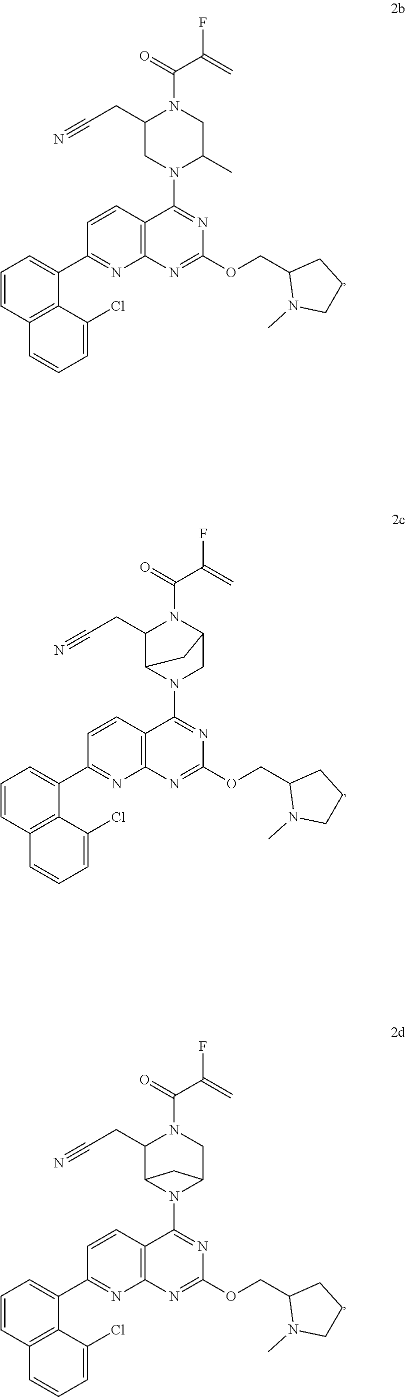 [SMILES: C=C(F)C(=O)N1CC(C)N(c2nc(OCC3CCCN3C)nc3nc(-c4cccc5cccc(Cl)c45)ccc23)CC1CC#N ; C=C(F)C(=O)N1C2CC(C1CC#N)N(c1nc(OCC3CCCN3C)nc3nc(-c4cccc5cccc(Cl)c45)ccc13)C2 ; C=C(F)C(=O)N1CC2CC(C1CC#N)N2c1nc(OCC2CCCN2C)nc2nc(-c3cccc4cccc(Cl)c34)ccc12]

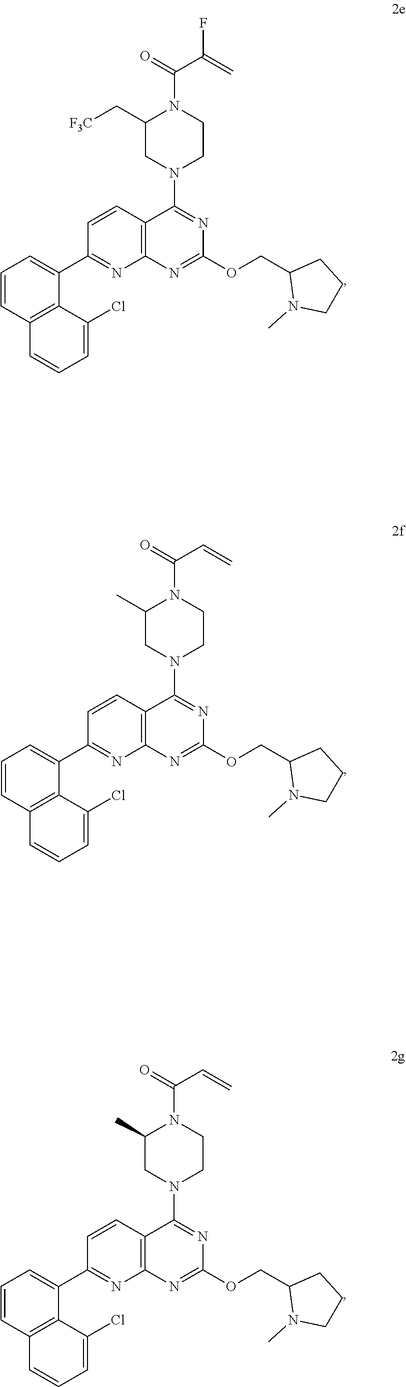 [SMILES: C=C(F)C(=O)N1CCN(c2nc(OCC3CCCN3C)nc3nc(-c4cccc5cccc(Cl)c45)ccc23)CC1CC(F)(F)F ; C=CC(=O)N1CCN(c2nc(OCC3CCCN3C)nc3nc(-c4cccc5cccc(Cl)c45)ccc23)CC1C ; C=CC(=O)N1CCN(c2nc(OCC3CCCN3C)nc3nc(-c4cccc5cccc(Cl)c45)ccc23)C[C@H]1C]

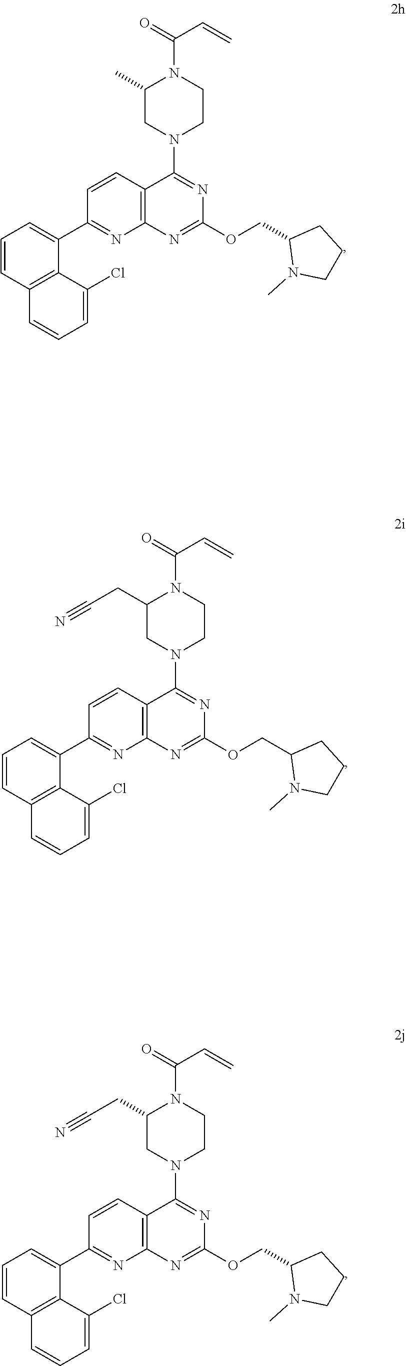

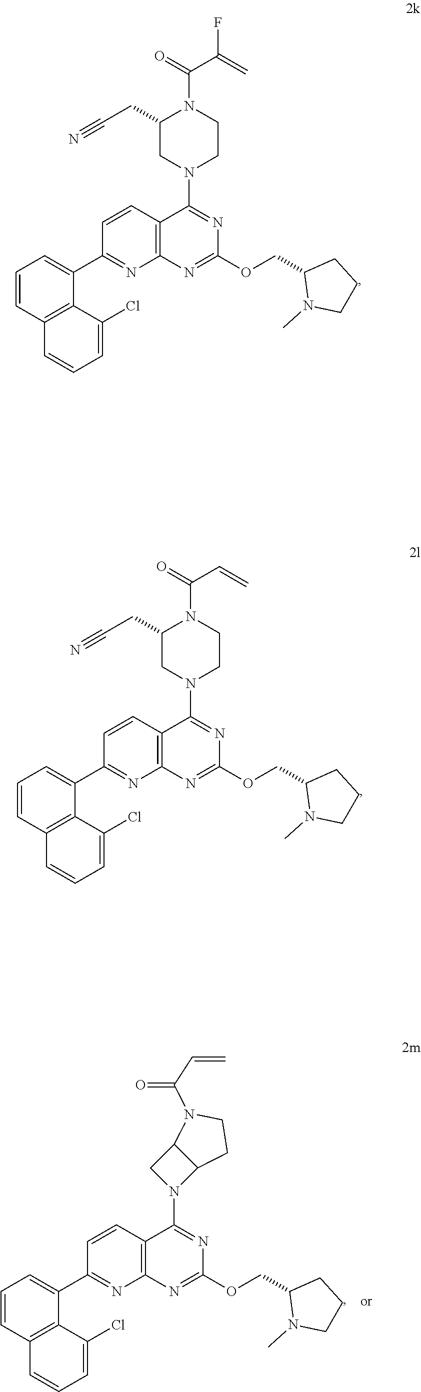

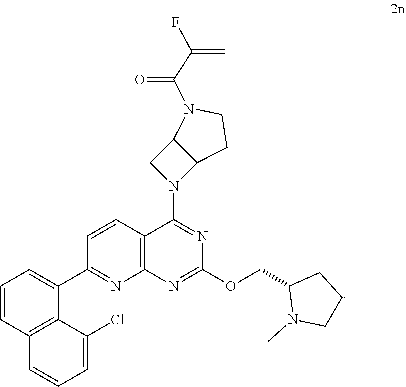

A particularly preferred compound of the invention is 2l:

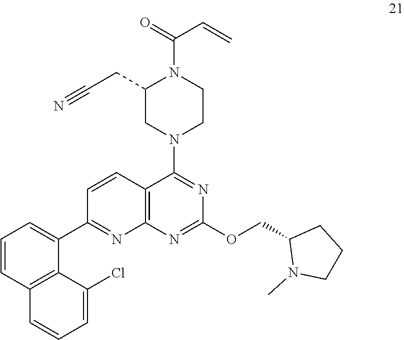 [SMILES: C=CC(=O)N1CCN(c2nc(OC[C@@H]3CCCN3C)nc3nc(-c4cccc5cccc(Cl)c45)ccc23)C[C@@H]1CC#N]

Compounds disclosed herein can be administered to a patient as the neat or freebase chemical, but are preferably administered as a pharmaceutical composition. Accordingly, the invention encompasses pharmaceutical compositions comprising a compound or a salt (including a pharmaceutically acceptable salt) of a compound, such as a compound of Formula I, together with at least one pharmaceutically acceptable carrier. The pharmaceutical composition may contain a compound or salt of Formula I as the only active agent, but is preferably contains at least one additional active agent. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition may also include a molar ratio of a compound, such as a compound of Formula I, and an additional active agent. For example, the pharmaceutical composition may contain a molar ratio of about 0.5:1, about 1:1, about 2:1, about 3:1 or from about 1.5:1 to about 4:1 of an additional active agent to a compound of Formula I.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the compound of the present invention.

The pharmaceutical compositions/combinations can be formulated for oral administration. These compositions contain between 0.1 and 99 weight % (wt %) of a compound of Formula III and usually at least about 5 wt % of a compound of Formula I. Some embodiments contain from about 25 wt % to about 50 wt % or from about 5 wt % to about 75 wt % of the compound of Formula I.

### Treatment Methods

The compounds of Formula I, as well as pharmaceutical compositions comprising the compounds, are useful for diagnosis or treatment of a disease, disorder, or medical condition mediated through KRAS, especially the KRAS mutant G12C, and including various cancers, such as glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.

According to the present invention, a method of KRAS-mediated diseases or conditions comprises providing to a patient in need of such treatment a therapeutically effective amount of a compound of Formula I. In one embodiment, the patient is a mammal, and more specifically a human. As will be understood by one skilled in the art, the invention also encompasses methods of treating non-human patients such as companion animals, e.g. cats, dogs, and livestock animals.

A therapeutically effective amount of a pharmaceutical composition is preferably an amount sufficient to reduce or ameliorate the symptoms of a disease or condition. In the case of KRAS-mediated diseases for example, a therapeutically effective amount may be an amount sufficient to reduce or ameliorate cancer. A therapeutically effective amount of a compound or pharmaceutical composition described herein will also provide a sufficient concentration of a compound of Formula I when administered to a patient. A sufficient concentration is preferably a concentration of the compound in the patient's body necessary to prevent or combat the disorder. Such an amount may be ascertained experimentally, for example by assaying blood concentration of the compound, or theoretically, by calculating bioavailability.

According to the invention, the methods of treatment disclosed herein include providing certain dosage amounts of a compound of Formula Ito a patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I are provided daily to a patient. Frequency of dosage may also vary depending on the compound used and the particular disease treated. However, for treatment of most KRAS-mediated diseases and disorders, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

A compound of Formula I may be administered singularly (i.e., sole therapeutic agent of a regime) to treat or prevent KRAS-mediated diseases and conditions such as various cancers, or may be administered in combination with another active agent. One or more compounds of Formula I may be administered in coordination with a regime of one or more other active agents such as anticancer cytotoxic agents. In an embodiment, a method of treating or diagnosing KRAS-mediated cancer in a mammal includes administering to said mammal a therapeutically effective amount of a compound of Formula I, optionally in combination with one or more additional active ingredients.

As will be appreciated by one skilled in the art, the methods of treatment provided herein are also useful for treatment of mammals other than humans, including for veterinary applications such as to treat horses and livestock, e.g. cattle, sheep, cows, goats, swine and the like, and pets (companion animals) such as dogs and cats.

For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g. blood, plasma, serum, cellular interstitial fluid, saliva, feces, and urine) and cell and tissue samples of the above subjects will be suitable for use.

In one embodiment, the invention provides a method of treating a disease, disorder, or medical condition mediated through KRAS, especially the KRAS mutant G12C, including various cancers, in a patient identified as in need of such treatment, the method comprising providing to the patient an effective amount of a compound of Formula I. The compounds of Formula I provided herein may be administered alone, or in combination with one or more other active agents.

In another embodiment, the method of treating or diagnosing KRAS-mediated diseases or conditions may additionally comprise administering the compound of Formula I in combination with one or more additional compounds, wherein at least one of the additional compounds is an active agent, to a patient in need of such treatment. The one or more additional compounds may include additional therapeutic compounds, including anticancer therapeutic compounds such as doxorubicin, paclitaxel, docetaxel, cisplatin, camptothecin, temozolomide, avastin, Herceptin, Erbitux, and the like.

### EXAMPLES

### Chemical Synthesis

The synthesis of compounds of this invention is exemplified by the sequence of steps shown in Schemes 1 and 2. In Scheme 1, sequential reaction of commercially available amide 3 with oxalyl chloride and ammonia in a solvent such as THF can afford 4. Reaction of 4 with a base such as lithium bis(trimethylsilyl)amide (LiHMDS) in a solvent such as THF will effect ring closure to yield compound 5. Reaction of 5 with an excess of a chlorinating agent such as phosphorus oxychloride will afford chloride 6. Reaction between compounds 6 and 7 in the presence of a base such as DIPEA in a solvent such as acetonitrile will produce 8. Treatment of 9a-9c with sodium hydride in a solvent such as N-methyl-2-pyrrolidone generates the corresponding anion of 9a-9c which reacts with 8 to afford compounds 10a-10c, respectively. Alternatively, 9b can be reacted with 8 in the presence of a base such as DIPEA to produce 10b. A standard Suzuki coupling procedure between compounds 10a-10c and 11 in a solvent mixture such as 1,4-dioxane and water can be employed to prepare compounds 12a-12c. Removal of the Boc protecting group of 12a-12c under acidic conditions such as anhydr. HCl in dioxane, followed by acylation of the deprotected product with an α,β-unsaturated acid chloride such as acryloyl chloride in a solvent such as methylene chloride containing a base such as triethylamine, will produce the corresponding compounds to generate compounds of the Formula I where X 13a-13c.

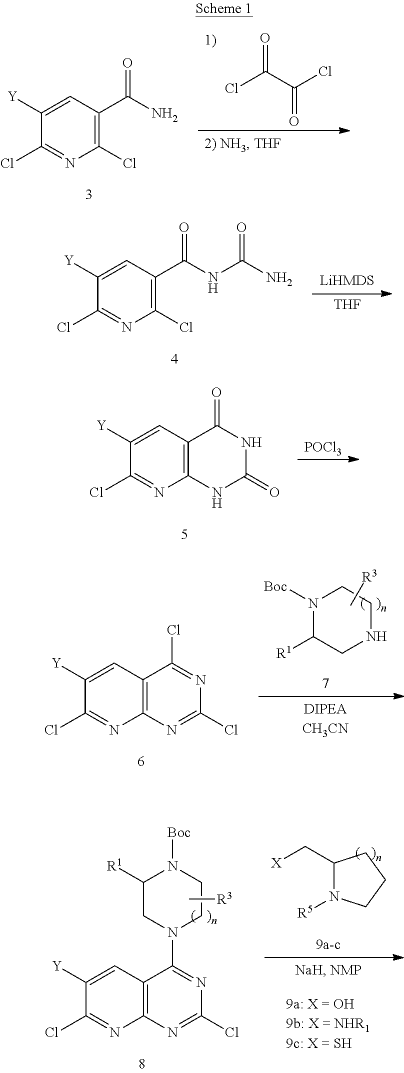

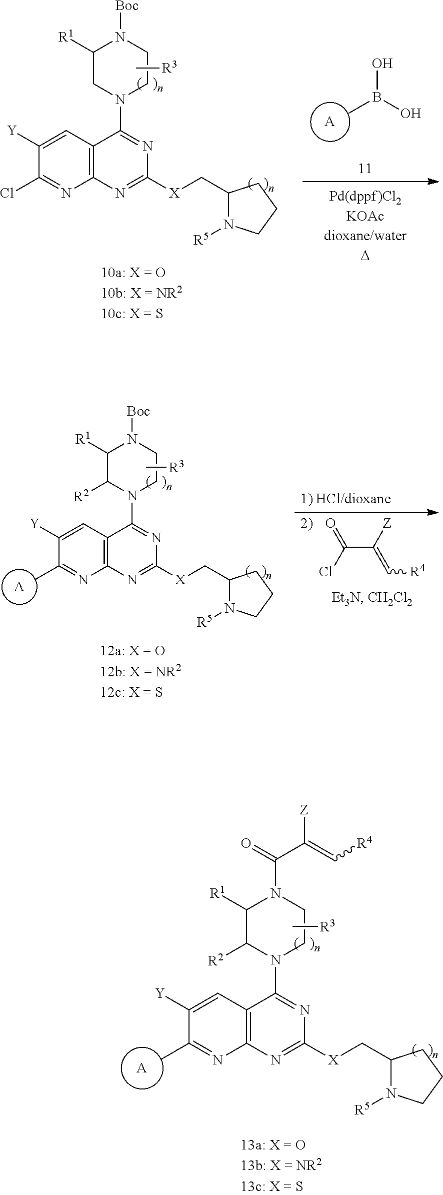

Similarly, Scheme 2 illustrates the synthesis of examples of the Formula I where X is methylene (14). The reaction of acetylene 15 with a strong base such as sodium hydride generates the corresponding acetylide anion, which can then be reacted with 8 to provide 16. Alternatively, Sonogashira coupling of 15 with 8 using a Pd catalyst such as Pd(dppf)₂Cl₂ can furnish compound 16. Catalytic hydrogenation of 16 followed by Suzuki coupling with boronic acid 11 generates compound 17. Removal of the Boc protecting group from 17 under acidic conditions such as TFA in dichloromethane affords the corresponding amine, which can be subsequently be reacted with an acryloyl chloride in a solvent such as methylene chloride containing a base such as triethylamine to generate compounds of the Formula I where X is methylene (14).

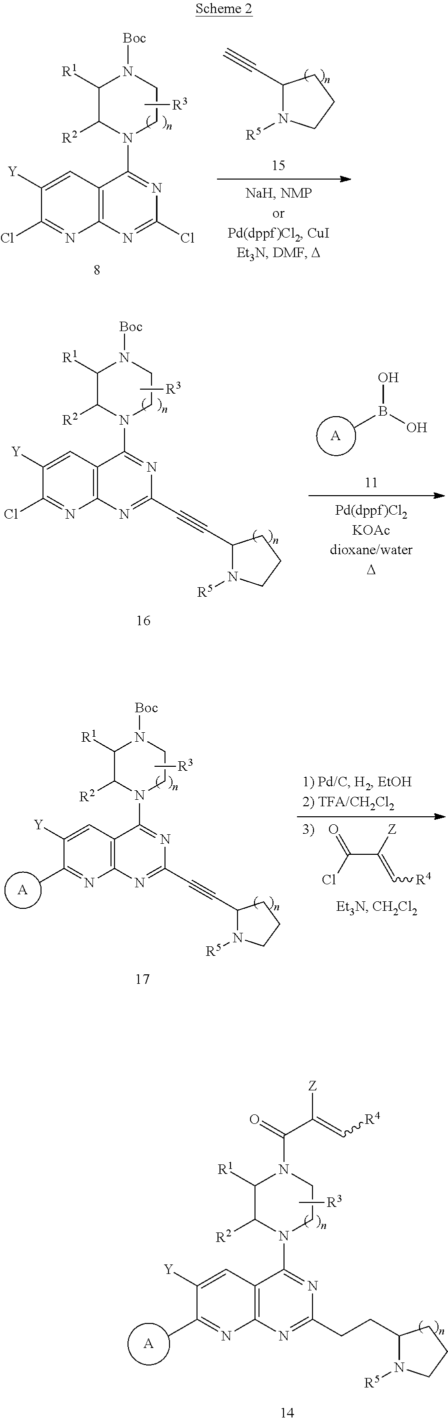

### Abbreviations and Acronyms

The following abbreviations and acronyms may be used in this application:

anhyd.=anhydrous;

aq.=aqueous;

B₂pin₂=bis(pinacolato)diboron;

Boc=tert-butoxycarbonyl;

n-Bu₃P=tri-n-butylphosphine;

Compd=compound;

d=day(s);

DCM=dichloromethane;

DIEA=DIPEA=N,N-diisopropylethylamine;

DMF=N,N-dimethylformamide;

DMSO=dimethylsulfoxide;

DMA=N,N-dimethylacetamide;

dppf=1,1′-bis(diphenylphosphino)ferrocene)

EtOAc=ethyl acetate;

equiv=equivalents;

Ex=Example;

h=hour(s);

LiHMDS=lithium bis(trimethylsilyl)amide [LiN(SiMe₃)₂];

MeOH=methanol;

NMP=N-methyl-2-pyrrolidone;

min=minutes;

Pd(dppf)Cl₂=[1,1′-bis(diphenylphosphino)ferrocene]dichloropalladium(II);

RT=room temperature;

satd.=saturated solution;

TEA=triethylamine;

TFA=trifluoroacetic acid;

THF=tetrahydrofuran;

### EXAMPLES

The present inventive concept has been described in terms of exemplary principles and embodiments, but those skilled in the art will recognize that variations may be made and equivalents substituted for what is described without departing from the scope and spirit of the disclosure as defined by the following claims.

### Example 1

### 1-[(2R)-4-[7-(8-Chloronaphthalen-1-yl)-2-[(1-methylpyrrolidin-2-yl)methoxy]pyrido[2,3-d]pyrimidin-4-yl]-2-methylpiperazin-1-yl]prop-2-en-1-one (18)

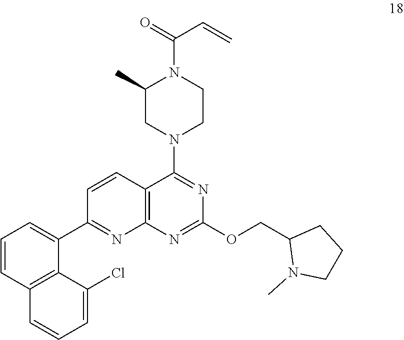 [SMILES: C=CC(=O)N1CCN(c2nc(OCC3CCCN3C)nc3nc(-c4cccc5cccc(Cl)c45)ccc23)C[C@H]1C]

Example 1 (18) was prepared as shown below in Scheme 3.

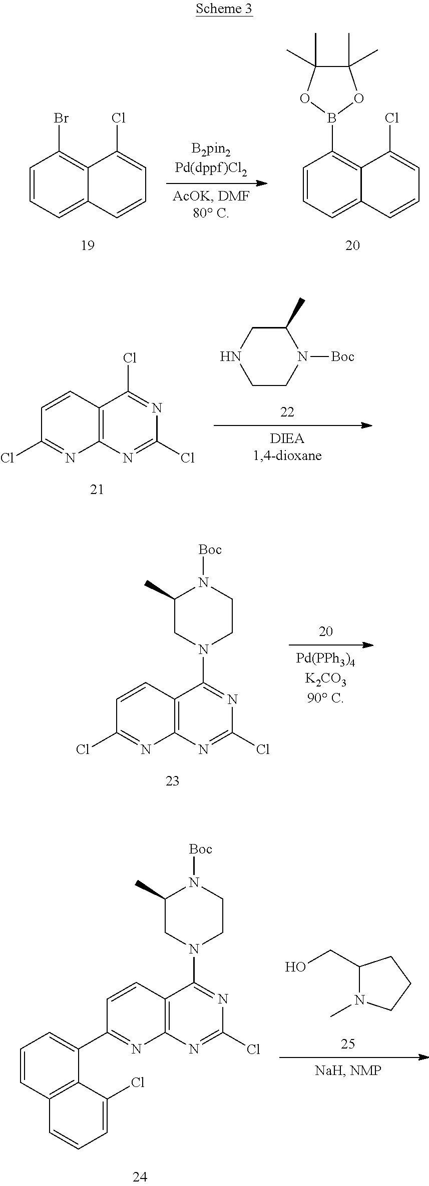

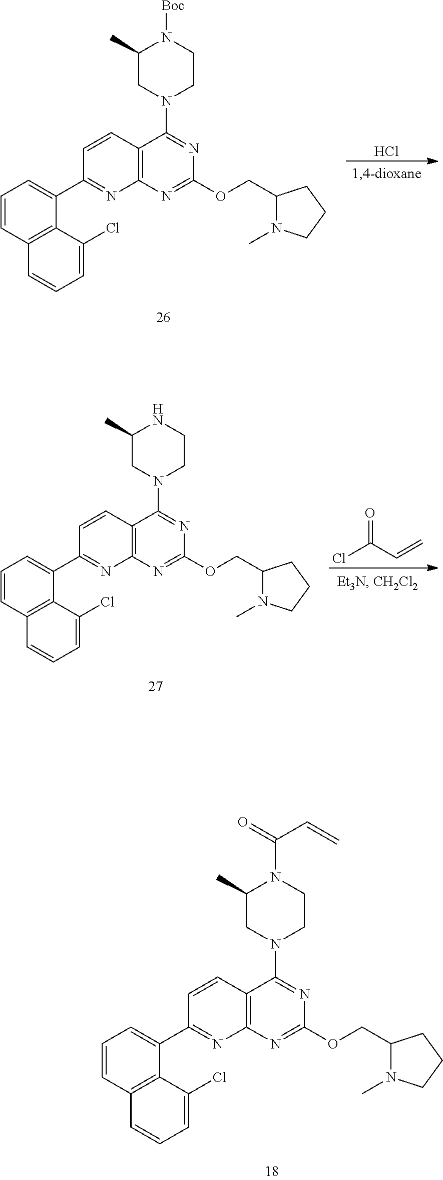

2-(8-Chloronaphthalen-1-yl)-4,4,5,5-tetramethyl-1,3,2-dioxaborolane (19). To a stirred degassed mixture of 1-bromo-8-chloronaphthalene (19; 5.00 g, 20.7 mmol, 1.00 equiv) and bis(pinacolato)diboron (5.78 g, 22.8 mmol, 1.10 equiv) in DMF (50 mL) were added Pd(dppf)Cl₂ (1.51 g, 2.064 mmol, 0.10 equiv) and potassium acetate (6.10 g, 62.1 mmol, 3.00 equiv) at RT under nitrogen atmosphere. The resulting mixture was heated at 80° C. while stirring overnight under a nitrogen atmosphere. The crude reaction mixture was then cooled to RT and concentrated under reduced pressure. The residue was purified by column chromatography on silica gel eluting with a mixture of petroleum ether/EtOAc (10:1) to afford 5.00 g (84%) of 2-(8-chloronaphthalen-1-yl)-4,4,5,5-tetramethyl-1,3,2-dioxaborolane (19) as a white solid: ¹H NMR (400 MHz, CDCl₃) δ 7.86 (dd, J=8.2, 1.3 Hz, 1H), 7.76 (dd, J=8.2, 1.2 Hz, 1H), 7.67 (dd, J=6.8, 1.3 Hz, 1H), 7.57 (dd, J=7.4, 1.3 Hz, 1H), 7.50 (dd, J=8.2, 6.8 Hz, 1H), 7.37 (dd, J=8.2, 7.4 Hz, 1H), 1.45 (s, 12H); ¹³C NMR (75 MHz, DMSO-d₆) δ 132.3, 131.8, 130.2, 128.8, 127.3, 126.6, 126.4, 84.2, 25.1.

tert-Butyl (2R)-4-[2,7-dichloropyrido[2,3-d]pyrimidin-4-yl]-2-methylpiperazine-1-carboxylate (23). A solution of 2,4,7-trichloropyrido[2,3-d]pyrimidine (21; 2.00 g, 8.53 mmol, 1.00 equiv) and DIEA (1.65 g, 12.8 mmol, 1.50 equiv) in 1,4-dioxane (50 mL) was treated portionwise with tert-butyl (2R)-2-methylpiperazine-1-carboxylate (22; 2.05 g, 10.3 mmol, 1.20 equiv) while stirring at RT. The resulting mixture was stirred for 1 h at RT and then concentrated under reduced pressure. The residue was purified by column chromatography on silica gel eluting with a mixture of petroleum ether/EtOAc (10:1) to afford 2.50 g (74%) of tert-butyl (2R)-4-[2,7-dichloropyrido[2,3-d]pyrimidin-4-yl]-2-methylpiperazine-1-carboxylate (23) as a yellow solid: HPLC-MS (ES⁺) m/z MH⁺=398.1.

tert-Butyl (2R)-4-[2-chloro-7-(8-chloronaphthalen-1-yl)pyrido[2,3-d]pyrimidin-4-yl]-2-methylpiperazine-1-carboxylate (24). To a degassed stirred solution of tert-butyl (2R)-4-[2,7-dichloropyrido[2,3-d]pyrimidin-4-yl]-2-methylpiperazine-1-carboxylate (23; 2.50 g, 6.28 mmol, 1.00 equiv) and 2-(8-chloronaphthalen-1-yl)-4,4,5,5-tetramethyl-1,3,2-dioxaborolane (20; 2.17 g, 7.53 mmol, 1.20 equiv) in dioxane (50 mL) was added K₂CO₃ (2.62 g, 18.8 mmol, 3.00 equiv) and tetrakis(triphenylphosphine)palladium(0) (0.73 g, 0.628 mmol, 0.10 equiv). The resulting mixture was heated at 90° C. overnight while stirring under a nitrogen atmosphere. The crude reaction mixture was concentrated in vacuo and the residue was purified by column chromatography on silica gel eluting with a mixture of petroleum ether/EtOAc (5:1) to afford 1.10 g (33%) of tert-butyl (2R)-4-[2-chloro-7-(8-chloronaphthalen-1-yl)pyrido[2,3-d]pyrimidin-4-yl]-2-methylpiperazine-1-carboxylate (24) as a yellow solid: HPLC-MS (ES⁺) m/z MH⁺=524.1.

tert-Butyl (2R)-4-[7-(8-chloronaphthalen-1-yl)-2-[(1-methylpyrrolidin-2-yl)methoxy]pyrido[2,3-d]pyrimidin-4-yl]-2-methylpiperazine-1-carboxylate (26). A mixture of tert-butyl (2R)-4-[2-chloro-7-(8-chloronaphthalen-1-yl)pyrido[2,3-d]pyrimidin-4-yl]-2-methylpiperazine-1-carboxylate (24; 500 mg, 0.953 mmol, 1.00 equiv) and NaH (60%, 76.3 mg, 1.91 mmol, 2.00 equiv) in NMP (5.0 mL) was treated dropwise with (1-methylpyrrolidin-2-yl)methanol (25; 132 mg, 1.14 mmol, 1.20 equiv) while stirring at 0° C. under nitrogen atmosphere. The resulting mixture was stirred for 2 h at RT under nitrogen atmosphere, cooled to 0° C. and then quenched with satd. aq. NH₄Cl (aq.). The crude reaction mixture was extracted with EtOAc (3×100 mL) and the combined organic extracts were washed with brine (3×100 mL), dried (Na₂SO₄), filtered and concentrate in vacuo. The residue was purified by column chromatography on silica gel eluting with petroleum ether/EtOAc (5:1) to afford 350 mg (61%) of tert-butyl (2R)-4-[7-(8-chloronaphthalen-1-yl)-2-[(1-methylpyrrolidin-2-yl)methoxy]pyrido[2,3-d]pyrimidin-4-yl]-2-methylpiperazine-1-carboxylate (26) as a yellow solid: HPLC-MS (ES⁺) m/z MH⁺=603.2.

7-(8-Chloronaphthalen-1-yl)-4-((R)-3-methylpiperazin-1-yl)-2-((1-methylpyrrolidin-2-yl)methoxy)pyrido[2,3-d]pyrimidine (27). A solution of tert-butyl (2R)-4-[7-(8-chloronaphthalen-1-yl)-2-[(1-methylpyrrolidin-2-yl)methoxy]pyrido[2,3-d]pyrimidin-4-yl]-2-methylpiperazine-1-carboxylate (26; 350 mg, 0.580 mmol, 1.00 equiv) in HCl (4M in dioxane, 15 mL) was stirred at RT for 2 h and then basified by the dropwise addition of satd. aq. NaHCO₃ at 0° C. The resulting mixture was extracted with ethyl acetate (3×100 mL), dried (Na₂SO₄), filtered and concentrated in vacuo. The crude product 7-(8-chloronaphthalen-1-yl)-4-((R)-3-methylpiperazin-1-yl)-2-((1-methylpyrrolidin-2-yl)methoxy)pyrido[2,3-d]pyrimidine (27) was used directly in the next step without further purification: HPLC-MS (ES⁺) m/z MH⁺=503.3.

1-[(2R)-4-[7-(8-Chloronaphthalen-1-yl)-2-[(1-methylpyrrolidin-2-yl)methoxy]pyrido[2,3-d]pyrimidin-4-yl]-2-methylpiperazin-1-yl]prop-2-en-1-one (18). To a mixture of (3R)-1-[7-(8-chloronaphthalen-1-yl)-2-[(1-methylpyrrolidin-2-yl)methoxy]pyrido[2,3-d]pyrimidin-4-yl]-3-methylpiperazine (27; 150 mg, 0.298 mmol, 1.00 equiv) and Et₃N (90.5 mg, 0.895 mmol, 3.00 equiv) in DCM (5.0 mL) was added acryloyl chloride (32.4 mg, 0.358 mmol, 1.20 equiv) dropwise at RT while stirring under a nitrogen atmosphere. After stirring at RT for 2 h, the reaction mixture was concentrated under vacuum and the crude product was purified by reversed phase Prep-HPLC on a Waters XBridge Prep C₁₈ OBD Column (130 Å, 5 μm, 19×150 mm) eluting with a gradient of 30-55% acetonitrile in water containing 0.01M NH₄HCO₃ to furnish 60 mg (36%) of 1-[(2R)-4-[7-(8-chloronaphthalen-1-yl)-2-[(1-methylpyrrolidin-2-yl)methoxy]pyrido[2,3-d]pyrimidin-4-yl]-2-methylpiperazin-1-yl]prop-2-en-1-one (18) as a white solid: HPLC-MS (ES+) m/z MH⁺=557.2; ¹H NMR (400 MHz, CD₃OD) δ 8.48 (dd, J=8.5, 4.3 Hz, 1H), 8.11-8.06 (m, 1H), 7.98 (dd, J=8.3, 2.8 Hz, 1H), 7.68-7.53 (m, 3H), 7.53-7.39 (m, 2H), 6.80 (m, 1H), 6.27 (m, 1H), 5.79 (m, 1H), 4.78-4.03 (m, 6H), 3.96-3.52 (m, 3H), 3.08 (m, 1H), 2.79 (m, 1H), 2.50 (m, 3H), 2.35 (m, 1H), 2.19-2.04 (m, 1H), 1.89-1.69 (m, 3H), 1.33 (m, 3H).

### Example 2

### 2-((S)-1-Acryloyl-4-(7-(8-chloronaphthalen-1-yl)-2-(((S)-1-methylpyrrolidin-2-yl)methoxy)pyrido[2,3-d]pyrimidin-4-yl)piperazin-2-yl)acetonitrile trifluoroacetate (1:1) (28)

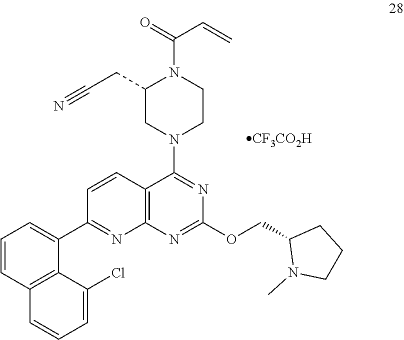 [SMILES: C=CC(=O)N1CCN(c2nc(OC[C@@H]3CCCN3C)nc3nc(-c4cccc5cccc(Cl)c45)ccc23)C[C@@H]1CC#N ; "·CF3CO2H"]

Example 2 (28, the trifluoroacetic acid salt of Compound 21 above) was prepared as shown below in Scheme 3.

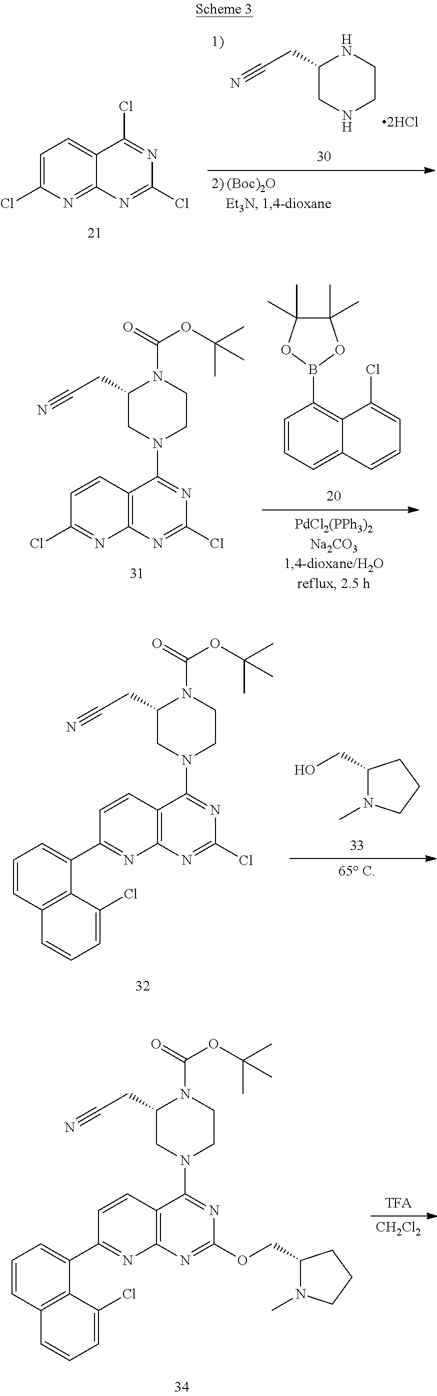

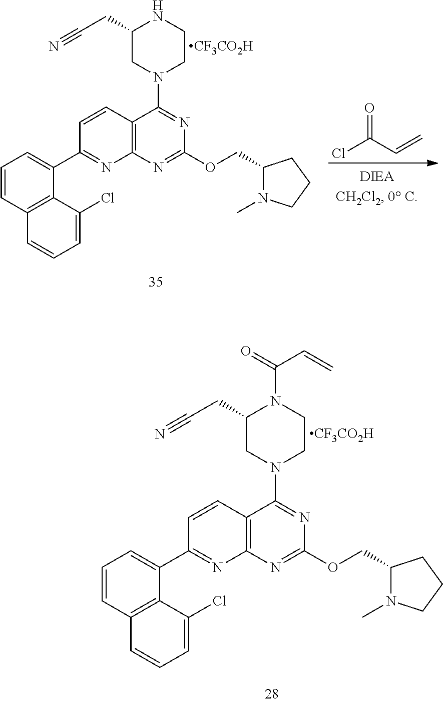

Tert-butyl (S)-2-(cyanomethyl)-4-(2,7-dichloropyrido[2,3-d]pyrimidin-4-yl)piperazine-1-carboxylate (31). Triethylamine (7.0 mL, 50 mmol) was added to a stirred suspension of (S)-2-(piperazin-2-yl)acetonitrile dihydrochloride (30; 1.98 g, 10.0 mmol) in anhyd. 1,4-dioxane (100 mL) and stirred to 2 h at RT under a nitrogen atmosphere. A solution of 2,4,7-trichloropyrido[2,3-d]pyrimidine (21; 2.34 g, 10.0 mmol) in anhyd. 1,4-dioxane (100 mL) was added dropwise over 30 min. The reaction mixture was stirred for an additional 30 min and subsequently treated with di-tert-butyl dicarbonate (3.50 mL, 15.2 mmol). After 30 min, additional di-tert-butyl dicarbonate (3.50 mL, 15.2 mmol) was added and the suspension was stirred at RT overnight. The reaction mixture was then partitioned between water and ethyl acetate and the organic extract washed with satd. aq. NaCl, dried (CaSO₄), filtered and concentrated in vacuo. The residue was purified by column chromatography on silica gel eluting with a gradient of 0-3% MeOH in DCM to provide 1.90 g of tert-butyl (S)-2-(cyanomethyl)-4-(2,7-dichloropyrido[2,3-d]pyrimidin-4-yl)piperazine-1-carboxylate (31) as an orange solid: HPLC-MS (ES⁺) m/z MH⁺=423; ¹H NMR (300 MHz, DMSO-d₆) δ 8.56 (d, J=8.7 Hz, 1H), 7.59 (d, J=8.7 Hz, 1H), 4.56 (m, 1H), 4.34-4.23 (m, 2H), 3.88-3.84 (m, 1H), 3.72-3.60 (m, 2H), 3.44 (m, 1H), 3.08-2.87 (m, 2H), 1.45 (s, 9H) ppm. ¹³C NMR (75 MHz, DMSO-d₆) δ 165.1, 161.0, 159.6, 156.0, 153.9, 139.9, 121.6, 118.7, 108.5, 80.6, 50.0, 48.7, 28.3, 19.6 ppm.

tert-Butyl (S)-4-(2-chloro-7-(8-chloronaphthalen-1-yl)pyrido[2,3-d]pyrimidin-4-yl)-2-(cyanomethyl)piperazine-1-carboxylate (32). Bis(triphenylphosphine)palladium(II) chloride (178 mg, 0.250 mmol) was added to a mixture of tert-butyl (S)-2-(cyanomethyl)-4-(2,7-dichloropyrido[2,3-d]pyrimidin-4-yl)piperazine-1-carboxylate (31; 1.09 g, 2.53 mmol), 2-(8-chloronaphthalen-1-yl)-4,4,5,5-tetramethyl-1,3,2-dioxaborolane (20; 877 mg, 3.04 mmol), and sodium carbonate (537 mg, 5.06 mmol) in 26 mL of water/1,4-dioxane (30% v/v). The rapidly stirred suspension was degassed at RT via 5 evacuation/N2 blanketing cycles and then heated at reflux under a N₂ atmosphere for 2.5 h. The reaction mixture was cooled to RT and partitioned between satd. aq. NaCl and ethyl acetate. The organic extract was dried (CaSO₄), filtered and concentrated in vacuo and the residue was purified by column chromatography on silica gel eluting with a gradient of 0-5% isopropanol in DCM to afford 492 mg of tert-butyl (S)-4-(2-chloro-7-(8-chloronaphthalen-1-yl)pyrido[2,3-d]pyrimidin-4-yl)-2-(cyanomethyl)piperazine-1-carboxylate (32) as a bright yellow solid: HPLC-MS (ES+) m/z MH⁺=549. ¹H NMR (300 MHz, DMSO-d6) δ 8.58 (d, J=8.5 Hz, 1H), 8.11 (d, J=7.9 Hz, 1H), 8.20 (d, J=8.0 Hz, 1H), 7.74-7.56 (m, 5H), 4.60 (m, 1H), 4.34-4.33 (m, 2H), 3.92-3.88 (m, 1H), 3.73-3.61 (m, 2H), 3.51 (m, 1H), 3.08-2.91 (m, 2H), 1.46 (s, 9H).

tert-Butyl (S)-4-(7-(8-chloronaphthalen-1-yl)-2-4(S)-1-methylpyrrolidin-2-yl)methoxy)pyrido[2,3-d]pyrimidin-4-yl)-2-(cyanomethyl)piperazine-1-carboxylate (34). (S)-(1-Methylpyrrolidin-2-yl)methanol (33; 1.00 mL, 8.40 mmol) was added to tert-butyl (S)-4-(2-chloro-7-(8-chloronaphthalen-1-yl)pyrido[2,3-d]pyrimidin-4-yl)-2-(cyanomethyl)piperazine-1-carboxylate (32; 143 mg, 0.260 mmol) the stirred neat mixture was heated at 65° C. for 2 h. The reaction mixture was cooled to RT and partitioned between satd. aq. NaHCO₃ and ethyl acetate and the organic extract was dried (CaSO₄), filtered and concentrated in vacuo. The residue was purified by column chromatography on silica gel eluting with a gradient of 0-10% MeOH in DCM to give 410 mg of tert-butyl (S)-4-(7-(8-chloronaphthalen-1-yl)-2-(((S)-1-methylpyrrolidin-2-yl)methoxy)pyrido[2,3-d]pyrimidin-4-yl)-2-(cyanomethyl)piperazine-1-carboxylate (34) as a light-yellow solid: HPLC-MS (ES⁺) m/z MH⁺=628.

2-((S)-4-(7-(8-Chloronaphthalen-1-yl)-2-(((S)-1-methylpyrrolidin-2-yl)methoxy)pyrido[2,3-d]pyrimidin-4-yl)piperazin-2-yl)acetonitrile trifluoroacetate (1:1) (35). Trifluoroacetic acid (0.90 mL, 12.0 mmol) was added to a stirred solution of tert-butyl (S)-4-(7-(8-chloronaphthalen-1-yl)-2-(((S)-1-methylpyrrolidin-2-yl)methoxy)pyrido[2,3-d]pyrimidin-4-yl)-2-(cyanomethyl)piperazine-1-carboxylate (34; 377 mg, 0.600 mmol) in anhydrous DCM (60 mL) at 0° C. The ice bath was removed and the reaction was stirred at RT for 5 h. The reaction mixture was washed with satd. aq. NaHCO₃, dried (CaSO₄), filtered and concentrated in vacuo to yield 399 mg of 2-((S)-4-(7-(8-chloronaphthalen-1-yl)-2-(((S)-1-methylpyrrolidin-2-yl)methoxy)pyrido[2,3-d]pyrimidin-4-yl)piperazin-2-yl)acetonitrile trifluoroacetate (1:1) (35) as a light-yellow solid which was used directly in the next step without further purification: HPLC-MS (ES⁺) m/z MH⁺=528.

2-((S)-1-Acryloyl-4-(7-(8-chloronaphthalen-1-yl)-2-(((S)-1-methylpyrrolidin-2-yl)methoxy)pyrido[2,3-d]pyrimidin-4-yl)piperazin-2-yl)acetonitrile trifluoroacetate (1:1) trifluoroacetate (1:1) (28). Acryloyl chloride (25 μL, 0.30 mmol) was added to a stirred solution of 2-((S)-4-(7-(8-chloronaphthalen-1-yl)-2-(((S)-1-methylpyrrolidin-2-yl)methoxy)pyrido[2,3-d]pyrimidin-4-yl)piperazin-2-yl)acetonitrile (35; 128 mg, 0.240 mmol) and diisopropylethylamine (0.20 mL, 1.21 mmol) in anhydrous DCM (10 mL) at 0° C. under a nitrogen atmosphere. After 30 min, the reaction mixture was concentrated in vacuo and the residue was purified by column chromatography on silica gel eluting with a gradient of 0-5% MeOH containing 2% NH4OH (v/v) in DCM to furnish 58 mg of 2-((S)-1-acryloyl-4-(7-(8-chloronaphthalen-1-yl)-2-(((S)-1-methylpyrrolidin-2-yl)methoxy)pyrido[2,3-d]pyrimidin yl)piperazin-2-yl)acetonitrile trifluoroacetate (1:1) (28) as a white solid. This material was subsequently recrystallized from and mixture of ethyl acetate and hexanes to yield 35 mg of 28 as a white powder: HPLC-MS (ES⁺) m/z MH⁺=582; ¹H NMR (300 MHz, DMSO-d₆) δ 10.57 (broad s, 1H), 8.54-8.51 (m, 1H), 8.20-8.17 (m, 1H), 8.11 (d, J=8.1 Hz, 1H), 7.74-7.65 (m, 2H), 7.61-7.50 (m, 3H), 6.96-6.80 (m, 1H), 6.21 (dd, J=16.63, 1.95 Hz, 1H), 5.80 (d, J=10.0 Hz, 1H), 4.98-4.88 (m, 1H), 4.74-4.62 (m, 2H), 4.35-4.32 (m, 2H), 4.11-3.44 (m, 6H), 3.14-3.06 (m, 3H), 2.94 (s, 3H), 2.32-2.21 (m, 1H), 2.09-1.76 (m, 3H).

### Nucleotide Exchange Assay

The biological activity of Examples 1 (18) and 2 (28) were determined in a KRAS G12C/SOS1 Nucleotide Exchange Assay that was performed by Reaction Biology Corporation (RBC), 1 Great Valley Parkway, Suite 2 Malvern, Pa. 19355, USA. The assay evaluates the SOS1-mediated Bodipy-GDP to GTP exchange observed with KRAS G12C.

The compounds were tested in 10 concentration IC50 mode with 3-fold serial dilution at a starting concentration of 10 μM for Examples 1 and 2 and 5 μM for the reference standard (ARS-1620). The compound pre-incubation time was 30 min at RT and the curve fits were performed when the activities at the highest concentration of compounds were less than 65%.

Reaction Buffer: 40 mM HEPES 7.4, 10 mM MgCl₂, 1 mM DTT 0.002% Triton X100, 0.1% DMSO (final).

Enzyme: SOS1 (RBC cat #MSC-11-502). Recombinant human SOS1 (Genbank accession #NM_005633.3; aa 564-1049, expressed in E. Coli with C-terminal StrepII. MW=60.59 kDa).

KRAS G12C: Recombinant human KRAS (Genbank accession #NM_033360.3; aa 2-169, expressed in E. coli with N-terminal TEV cleavable his-tag. MW 21.4 kDa) KRAS is pre-loaded with Bodipy-GDP.

Final concentrations: KRAS-bodipy-GDP was 0.125 μM; SOS1 was 70 nM; and GTP was 25 μM. In addition, the final assay volume was 15 μL.

Reaction Procedure:
1. Deliver 10 uL of 1.5× KRAS solution in freshly prepared reaction buffer to reaction wells.
2. Deliver compounds in 100% DMSO into buffer using acoustic technology (Echo550; nanoliter range).
3. Incubate compounds with Kras for 30 minutes at room temperature.
4. Prepare 3× (SOS1+GTP) solution in reaction buffer.
5. Deliver 5 μL of SOS1+GTP solution into reactions wells (deliver GTP only to column 1 for no SOS1 control).
6. Monitor reaction progress for 30 minutes at RT using a Clariostar plate reader (ex 483-14, ems 530-30).

Data Analysis: The fluorescence data was normalized using the equation below and fitted to “one phase exponential decay” equation using GraphPad prism software. The plateau was fixed to zero (use for non-covalent inhibitors) and rate ×1000 was used to calculate the IC₅₀ values. Alternatively, the plateau was unconstrained (for covalent inhibitors) and the span value was used to calculate the IC₅₀ values.

$$Ynorm = 1 - \frac{( Yraw - Ao )}{( M - Ao )}$$

where Yraw is defined as fluorescence at time t, Ao is the average initial fluorescence with no SOS1, and M is the minimum fluorescence at the end of the reaction at the maximum SOS1.

The background subtracted signals (no SOS1 protein wells were used as background) were converted to % activity relative to DMSO controls. Data was analyzed using GraphPad Prism 4 with “sigmoidal dose-response (variable slope)”; 4 parameters with Hill Slope. The constraints were bottom (constant equal to 0) and top (must be less than 1).

Results:

KRAS G12C*

| Analysis Method | Rate Constant | Span |
|---|---|---|
| Compound | IC₅₀ (nM) | IC₅₀ (nM) |
| ARS-1620 | 37 | 120 |
| MRTX-849 | 10 | 22 |
| AMG-510 | 7 | 25 |
| Example 1 | 58 | <65% inhibition |
| (Compound 18) | | |
| Example 2 | 8 | 25 |
| (Compound 28) | | |

*The substrate was Bodipy-GDP/Kras G12C with 0.5% DMSO added to the reaction. ARS-1620, MRTX-849 and AMG-510 are reference standards.

1. A compound of Formula I: 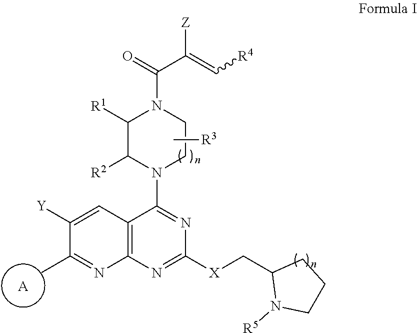
or a salt, solvate, or prodrug thereof, wherein
A is chosen from aryl or heteroaryl optionally substituted with one or more of hydrogen, halogen, hydroxyl, C₁₋₆alkyl, C₂₋₆alkenyl, C₂₋₆alkynyl, —(C₀₋₆alkyl)cycloalkyl, C₁₋₆haloalkyl, C₁₋₆ alkoxy, NO₂, cyano, CO₂H, PO(R⁶)₂, NH₂, NH(C₁₋₆alkyl) or N(C₁₋₆alkyl)₂;
X is chosen from O, S or CH₂;
Y is chosen from hydrogen, halogen or trifluoromethyl;
Z is chosen from hydrogen, halogen, trifluoromethyl or C₁₋₆alkyl;
R¹ is chosen from hydrogen, C₁₋₆alkyl, —(C₁₋₆alkyl)C₁₋₆alkoxy, —C₁₋₆alkyl(cycloalkyl), C₁₋₆aloalkyl, —(C₁₋₆alkyl)CN or —(C₁₋₆alkyl)P(O)(R⁶)₂;
R² is chosen from hydrogen, C₁₋₆alkyl, —(C₁₋₆alkyl)C₁₋₆alkoxy, —C₀₋₆alkyl(cycloalkyl), C₁₋₆haloalkyl, —(C₁₋₆alkyl)CN, —(C₁₋₆alkyl)P(O)(R⁶)₂ or taken together with R³ to represent 3-6 membered bridged ring;
R³ is chosen from hydrogen, C₁₋₆alkyl, —(C₁₋₆alkyl)C₁₋₆alkoxy, —C₀₋₆alkyl(cycloalkyl), C₁₋₆haloalkyl, —(C₁₋₆alkyl)CN, —(C₁₋₆alkyl)P(O)(R⁶)₂ or taken together with R² to represent 3-6 membered bridged alkyl ring;
n is 1-3;
R⁴ is chosen from hydrogen, halogen, C₁₋₃alkyl, C₁₋₃haloalkyl, —(C₁₋₃alkyl)CF₃ or —(C₁₋₃alkyl)C₁₋₆alkoxy;
R⁵ is chosen from H, C₁₋₆alkyl, C₃₋₆cycloalkyl or —(C₁₋₆alkyl)P(O)(R₆)₂;
R⁶ is chosen from H, C₁₋₆alkyl, C₃₋₆cycloalkyl, C₁₋₆alkoxy, C₃₋₆cycloalkoxy, hydroxyl, aryl or aryloxy;
R⁷ is chosen from H, C₁₋₆ alkyl, or C₃₋₆cycloalkyl.

2. The compound of claim 1 wherein the compound of Formula I is selected from compound 1a-1z or 2a-2n or a salt, solvate, or prodrug thereof:

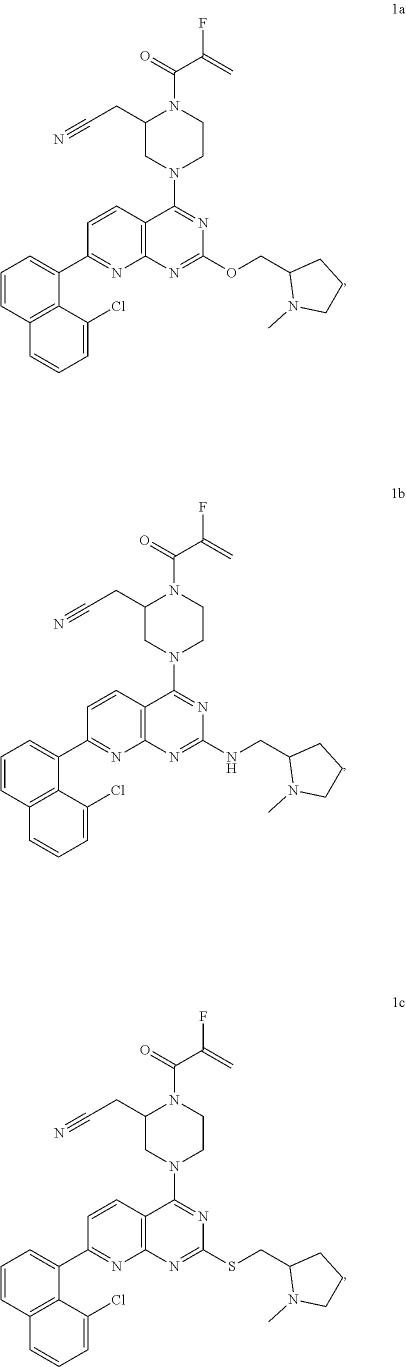 [SMILES: C=C(F)C(=O)N1CCN(c2nc(OCC3CCCN3C)nc3nc(-c4cccc5cccc(Cl)c45)ccc23)CC1CC#N ; C=C(F)C(=O)N1CCN(c2nc(NCC3CCCN3C)nc3nc(-c4cccc5cccc(Cl)c45)ccc23)CC1CC#N ; C=C(F)C(=O)N1CCN(c2nc(SCC3CCCN3C)nc3nc(-c4cccc5cccc(Cl)c45)ccc23)CC1CC#N]

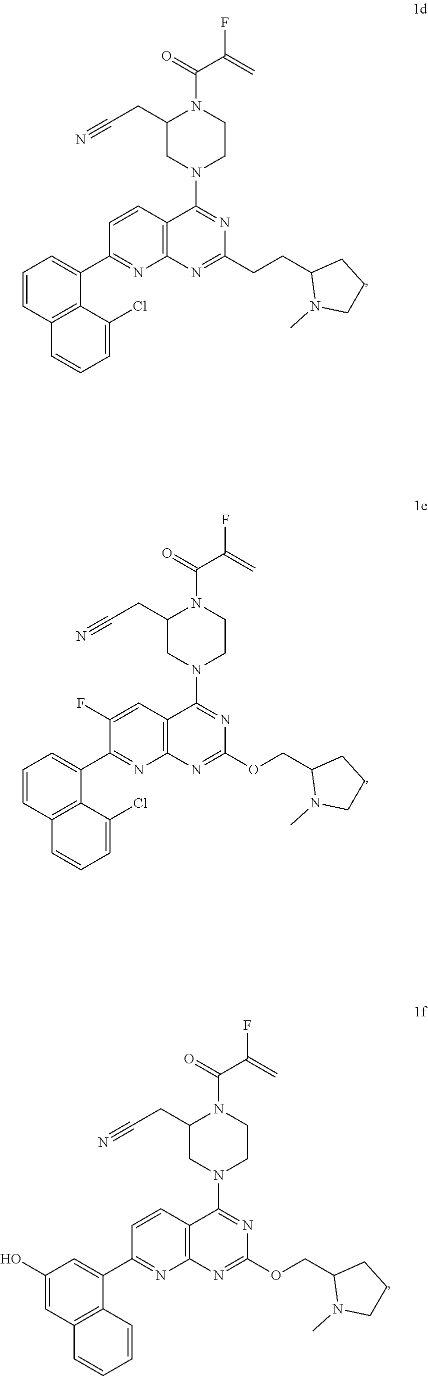 [SMILES: C=C(F)C(=O)N1CCN(c2nc(CCC3CCCN3C)nc3nc(-c4cccc5cccc(Cl)c45)ccc23)CC1CC#N ; C=C(F)C(=O)N1CCN(c2nc(OCC3CCCN3C)nc3nc(-c4cccc5cccc(Cl)c45)c(F)cc23)CC1CC#N ; C=C(F)C(=O)N1CCN(c2nc(OCC3CCCN3C)nc3nc(-c4cc(O)cc5ccccc45)ccc23)CC1CC#N]

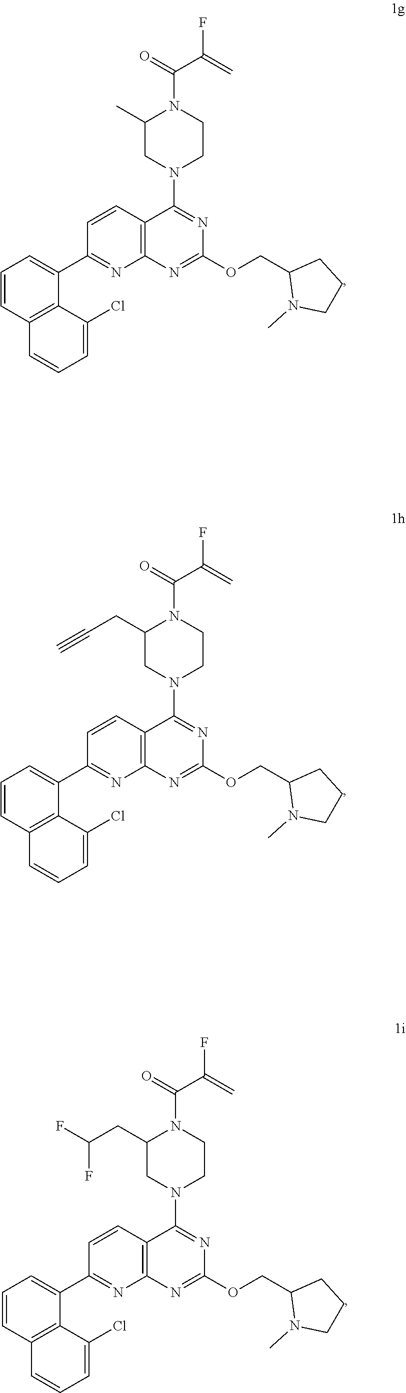 [SMILES: C=C(F)C(=O)N1CCN(c2nc(OCC3CCCN3C)nc3nc(-c4cccc5cccc(Cl)c45)ccc23)CC1C ; C#CCC1CN(c2nc(OCC3CCCN3C)nc3nc(-c4cccc5cccc(Cl)c45)ccc23)CCN1C(=O)C(=C)F ; C=C(F)C(=O)N1CCN(c2nc(OCC3CCCN3C)nc3nc(-c4cccc5cccc(Cl)c45)ccc23)CC1CC(F)F]

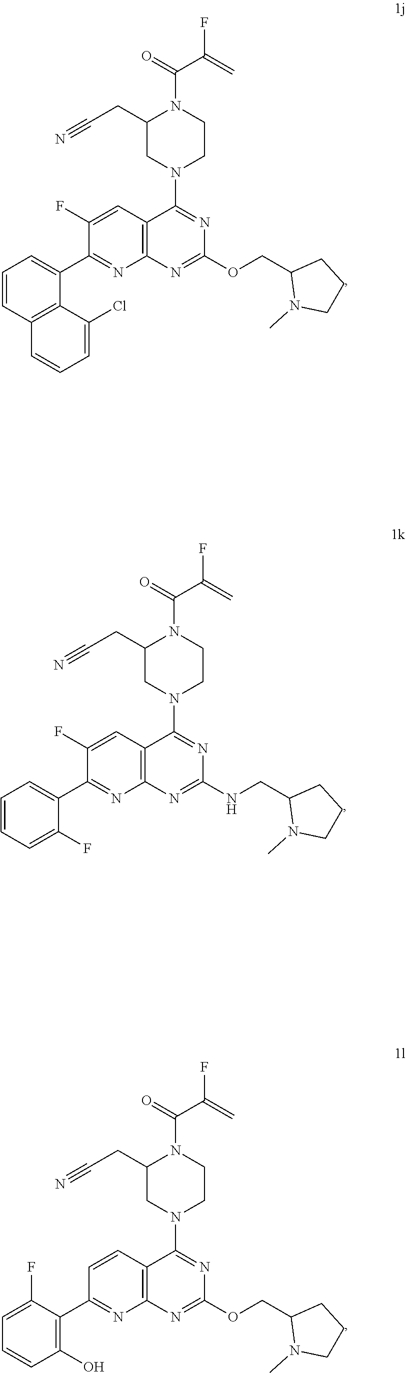 [SMILES: C=C(F)C(=O)N1CCN(c2nc(OCC3CCCN3C)nc3nc(-c4cccc5cccc(Cl)c45)c(F)cc23)CC1CC#N ; C=C(F)C(=O)N1CCN(c2nc(NCC3CCCN3C)nc3nc(-c4ccccc4F)c(F)cc23)CC1CC#N ; C=C(F)C(=O)N1CCN(c2nc(OCC3CCCN3C)nc3nc(-c4c(O)cccc4F)ccc23)CC1CC#N]

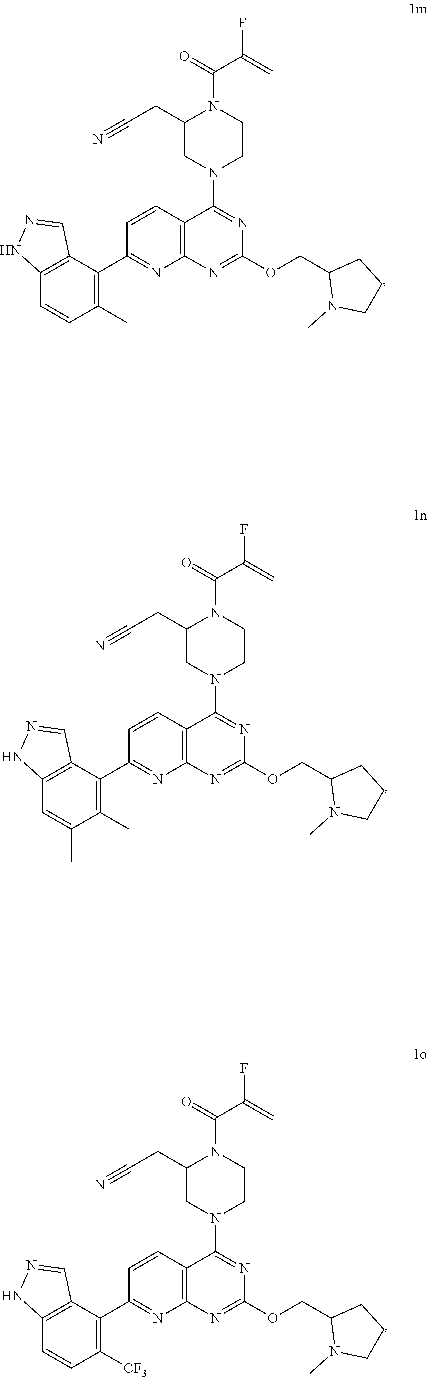 [SMILES: C=C(F)C(=O)N1CCN(c2nc(OCC3CCCN3C)nc3nc(-c4c(C)ccc5[nH]ncc45)ccc23)CC1CC#N ; C=C(F)C(=O)N1CCN(c2nc(OCC3CCCN3C)nc3nc(-c4c(C)c(C)cc5[nH]ncc45)ccc23)CC1CC#N ; C=C(F)C(=O)N1CCN(c2nc(OCC3CCCN3C)nc3nc(-c4c(C(F)(F)F)ccc5[nH]ncc45)ccc23)CC1CC#N]

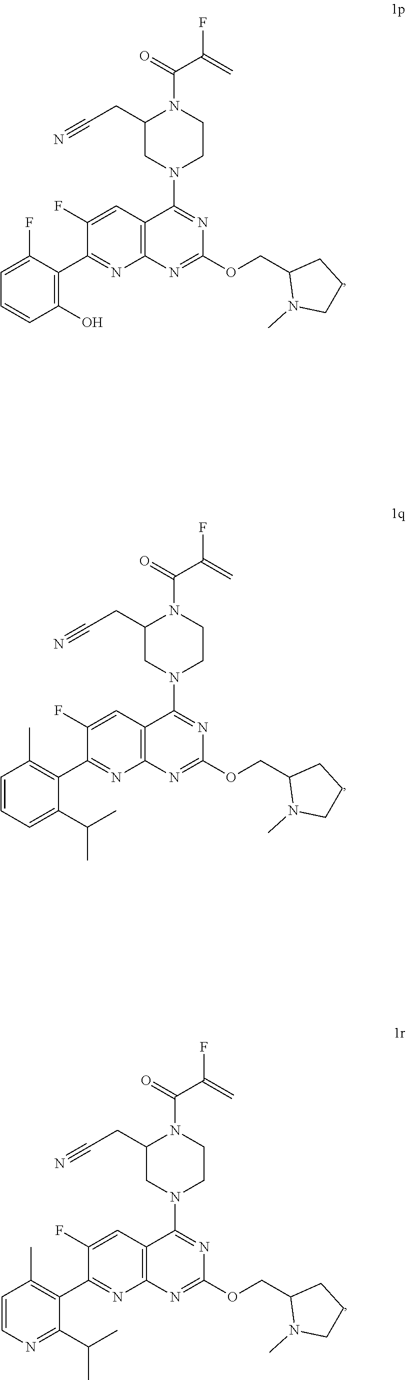 [SMILES: C=C(F)C(=O)N1CCN(c2nc(OCC3CCCN3C)nc3nc(-c4c(O)cccc4F)c(F)cc23)CC1CC#N ; C=C(F)C(=O)N1CCN(c2nc(OCC3CCCN3C)nc3nc(-c4c(C)cccc4C(C)C)c(F)cc23)CC1CC#N ; C=C(F)C(=O)N1CCN(c2nc(OCC3CCCN3C)nc3nc(-c4c(C)ccnc4C(C)C)c(F)cc23)CC1CC#N]

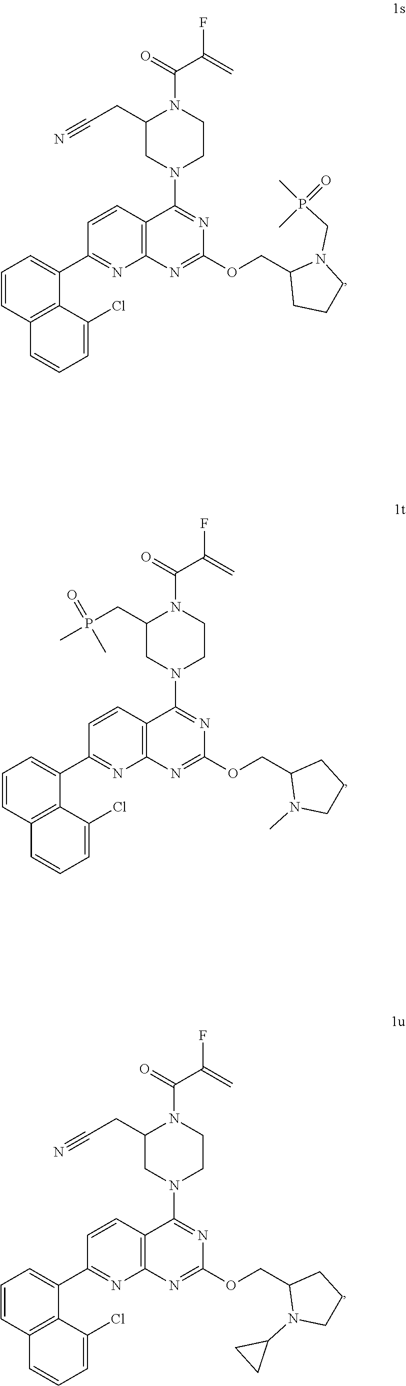 [SMILES: C=C(F)C(=O)N1CCN(c2nc(OCC3CCCN3CP(C)(C)=O)nc3nc(-c4cccc5cccc(Cl)c45)ccc23)CC1CC#N ; C=C(F)C(=O)N1CCN(c2nc(OCC3CCCN3C)nc3nc(-c4cccc5cccc(Cl)c45)ccc23)CC1CP(C)(C)=O ; C=C(F)C(=O)N1CCN(c2nc(OCC3CCCN3C3CC3)nc3nc(-c4cccc5cccc(Cl)c45)ccc23)CC1CC#N]

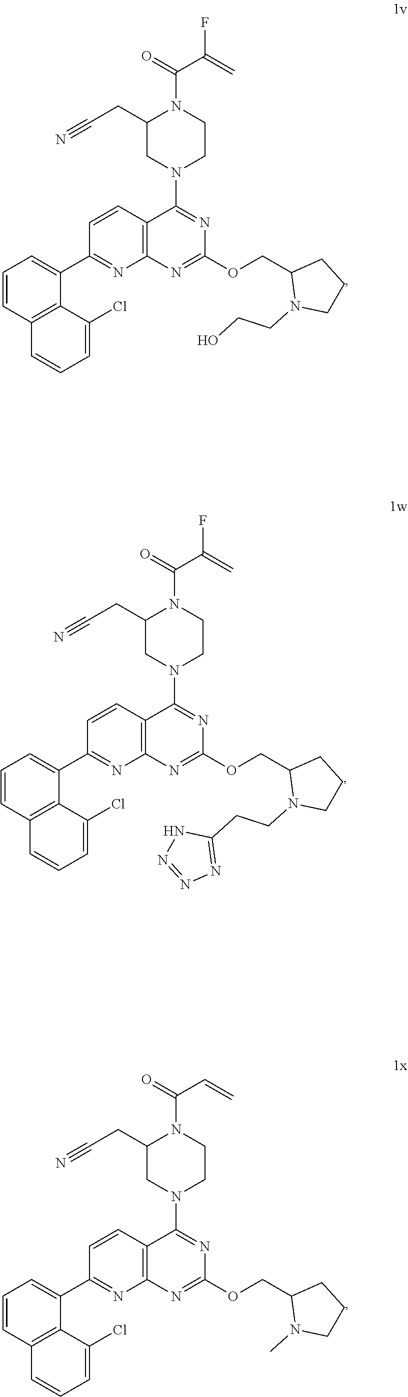 [SMILES: C=C(F)C(=O)N1CCN(c2nc(OCC3CCCN3CCO)nc3nc(-c4cccc5cccc(Cl)c45)ccc23)CC1CC#N ; C=C(F)C(=O)N1CCN(c2nc(OCC3CCCN3CCc3nnn[nH]3)nc3nc(-c4cccc5cccc(Cl)c45)ccc23)CC1CC#N ; C=CC(=O)N1CCN(c2nc(OCC3CCCN3C)nc3nc(-c4cccc5cccc(Cl)c45)ccc23)CC1CC#N]

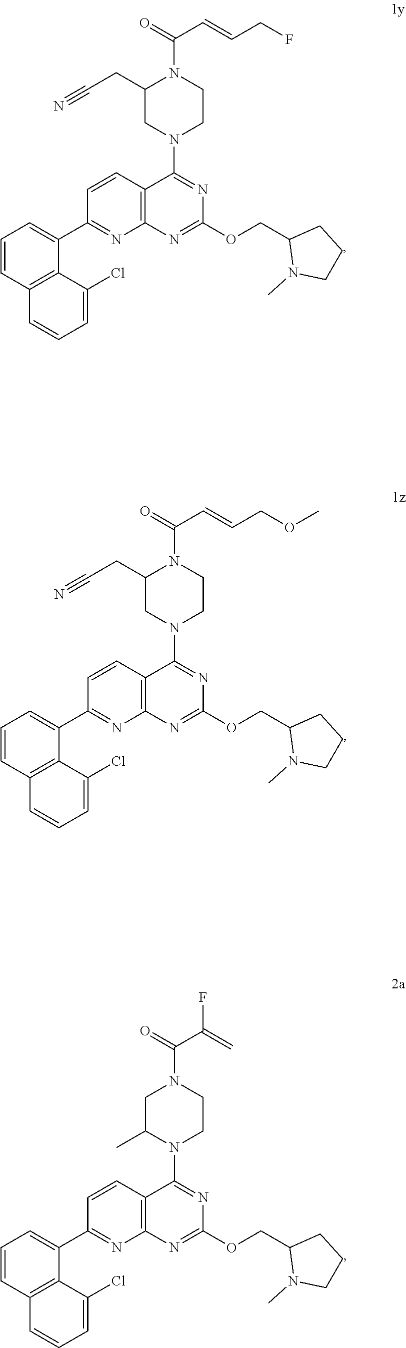 [SMILES: CN1CCCC1COc1nc(N2CCN(C(=O)/C=C/CF)C(CC#N)C2)c2ccc(-c3cccc4cccc(Cl)c34)nc2n1 ; COC/C=C/C(=O)N1CCN(c2nc(OCC3CCCN3C)nc3nc(-c4cccc5cccc(Cl)c45)ccc23)CC1CC#N ; C=C(F)C(=O)N1CCN(c2nc(OCC3CCCN3C)nc3nc(-c4cccc5cccc(Cl)c45)ccc23)C(C)C1]

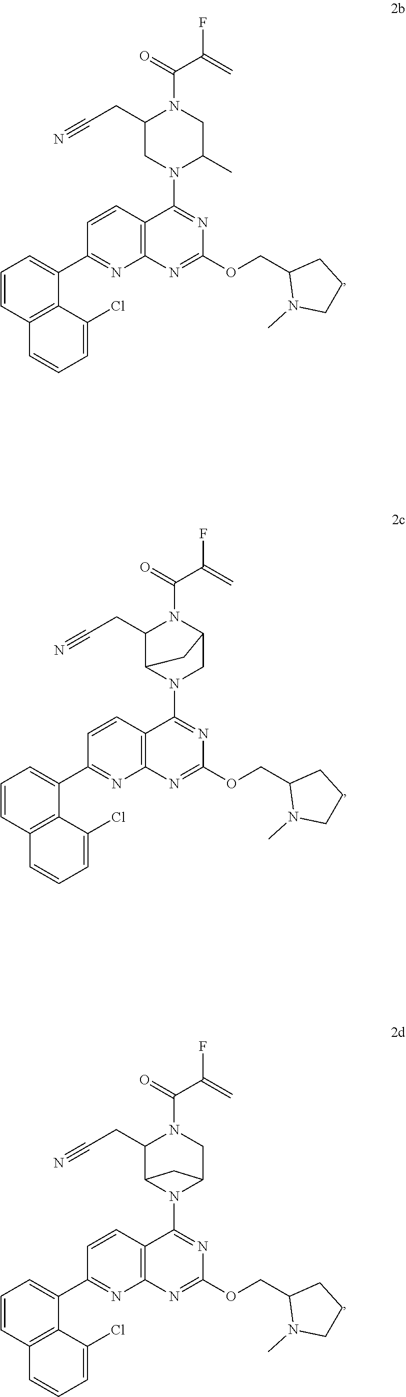 [SMILES: C=C(F)C(=O)N1CC(C)N(c2nc(OCC3CCCN3C)nc3nc(-c4cccc5cccc(Cl)c45)ccc23)CC1CC#N ; C=C(F)C(=O)N1C2CC(C1CC#N)N(c1nc(OCC3CCCN3C)nc3nc(-c4cccc5cccc(Cl)c45)ccc13)C2 ; C=C(F)C(=O)N1CC2CC(C1CC#N)N2c1nc(OCC2CCCN2C)nc2nc(-c3cccc4cccc(Cl)c34)ccc12]

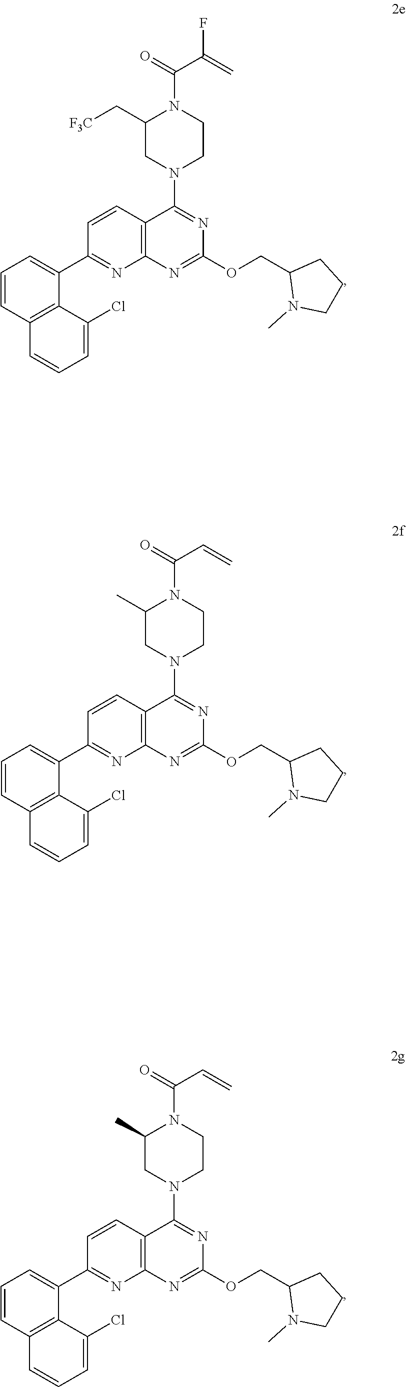 [SMILES: C=C(F)C(=O)N1CCN(c2nc(OCC3CCCN3C)nc3nc(-c4cccc5cccc(Cl)c45)ccc23)CC1CC(F)(F)F ; C=CC(=O)N1CCN(c2nc(OCC3CCCN3C)nc3nc(-c4cccc5cccc(Cl)c45)ccc23)CC1C ; C=CC(=O)N1CCN(c2nc(OCC3CCCN3C)nc3nc(-c4cccc5cccc(Cl)c45)ccc23)C[C@H]1C]

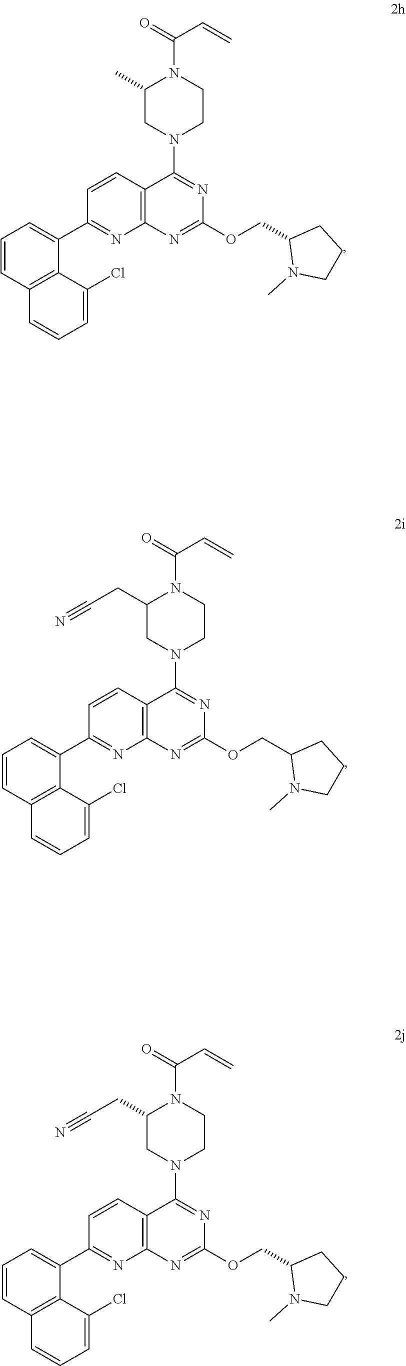

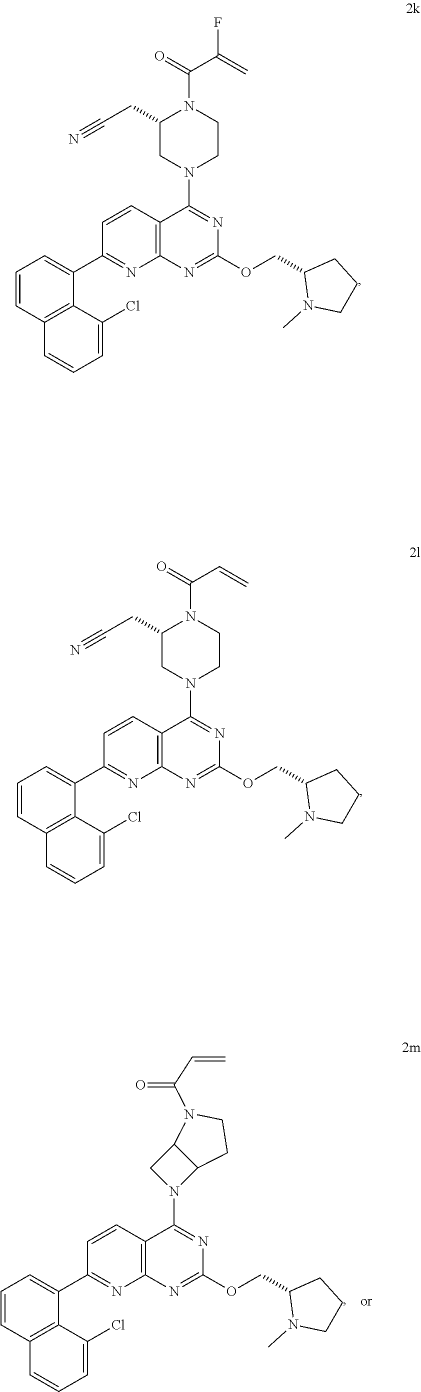

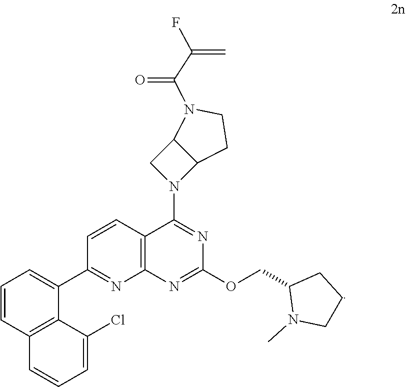

3. A pharmaceutical composition comprising the compound of claim 1, or a salt, solvate, or prodrug thereof, together with a pharmaceutically acceptable carrier.

4. A method of treating a disease, disorder, or medical condition in a patient, comprising the step of providing to a patient in need thereof a therapeutic agent, wherein the therapeutic agent is a compound of claim 1, or a salt, solvate, or prodrug thereof

5. The method of treating a disease, disorder, or medical condition in claim 4 in a patient, wherein said diseases include various cancers.

6. The method of treating a disease, disorder, or medical condition of claim 5 in a patient, wherein said disease, disorder, or medical condition is mediated through KRAS.

7. The method of treating a disease, disorder, or medical condition of claim 6 in a patient, wherein said disease, disorder, or medical condition is mediated through the KRAS mutant G12C.

8. The method of claim 5, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.

9. The method of claim 4, further comprising administering to the patient in need thereof at least one additional therapeutic agent.

10. The compound of claim 2 wherein the compound of Formula I is compound 2l or a salt, solvate, or prodrug thereof

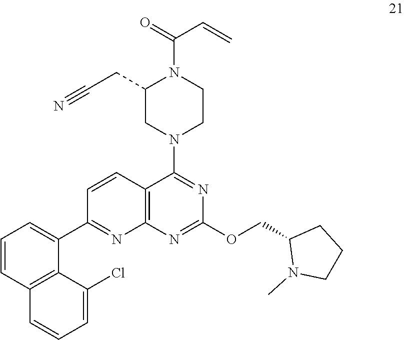 [SMILES: C=CC(=O)N1CCN(c2nc(OC[C@@H]3CCCN3C)nc3nc(-c4cccc5cccc(Cl)c45)ccc23)C[C@@H]1CC#N]

11. The pharmaceutical composition of claim 3, comprising Compound 2l or a salt, solvate, or prodrug thereof together with a pharmaceutically acceptable carrier.

12. The method of claim 4, comprising the step of providing to a patient in need thereof a pharmaceutical composition, comprising Compound 2l or a salt, solvate, or prodrug thereof together with a pharmaceutically acceptable carrier.

13. The method of claim 12, wherein said diseases include various cancers.

14. The method of claim 12, wherein said disease, disorder, or medical condition is mediated through KRAS.

15. The method of claim 14, wherein said disease, disorder, or medical condition is mediated through KRAS mutant G12C.

16. The method of claim 13, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.
